(19) United States
(12) Patent Application Publication
(10) Pub. No.: US 2012/0220832 A1
(43) Pub. Date: Aug. 30, 2012

(54) ENDOSCOPE BENDING DEVICE

(75) Inventors: Toshihiko NAKADE, Hachioji-shi (JP); Nobuyuki Matsuura, Hino-shi (JP); Yoshiyuki Tanii, Hamura-shi (JP)

(73) Assignees: OLYMPUS MEDICAL SYSTEMS CORP., Tokyo (JP); OLYMPUS CORPORATION, Tokyo (JP)

(21) Appl. No.: 13/466,430

(22) Filed: May 8, 2012

Related U.S. Application Data
(63) Continuation of application No. PCT/JP2010/066981, filed on Sep. 29, 2010.

(30) Foreign Application Priority Data
Jan. 13, 2010 (JP) 2010-005321

(51) Int. Cl. A61B 1/01

(52) U.S. Cl. 600/149

(57) ABSTRACT

An endoscope bending device includes an operation wire which includes a wire proximal end movably provided in an inner peripheral groove of a pulley, and a wire distal end connected to a bending section, the operation wire extending into an endoscope insertion section after being wound around an outer peripheral groove or the inner peripheral groove in a neutral condition in which the bending section is not bent. The endoscope bending device includes a slack absorbing portion which is configured to absorb the slack of the operation wire by moving the wire proximal end of the operation wire in the inner peripheral groove in a direction opposite to a discharging direction of the operation wire during the operation of discharging the operation wire from the neutral condition.

### CROSS-REFERENCE TO RELATED APPLICATIONS

This application is a Continuation Application of PCT Application No. PCT/JP2010/066981, filed Sep. 29, 2010 and based upon and claiming the benefit of priority from prior Japanese Patent Application No. 2010-005321, filed Jan. 13, 2010, the entire contents of all of which are incorporated herein by reference.

### BACKGROUND OF THE INVENTION

1. Field of the Invention

The present invention relates to an endoscope bending device which performs a bending operation in a bending operation section of an operation section of an endoscope to bend a bending section of an insertion section of the endoscope in a desired direction.

2. Description of the Related Art

In general, an endoscope includes an elongate insertion section configured to be inserted into a body cavity, and an operation section provided to a proximal side of the insertion section. The insertion section includes an elongate flexible section having flexibility, a bending section which configured to bend and which is provided to a distal side of the flexible section, and a distal rigid section provided to the distal side of the bending section. The endoscope is also provided with an endoscope bending device which configured to bend the bending section.

The endoscope bending device includes a bending operation section such as a bending operation knob provided in an operation section casing of the operation section. The bending operation section is coupled to a bending operation transmission mechanism provided, in the operation section casing. In the insertion section, operation wires longitudinally extend. The bending operation transmission mechanism includes a pulley to which proximal ends of the pair of operation wires are fixed. The pulley is rotated about an axis by the bending operation in the bending operation section. As a result of the rotation of the pulley, one of the pair of operation wires is wound around the pulley, and the other operation wire is discharged from pulley. The bending section bends due to the winding and discharging of the operation wires. When the rotation direction of the pulley is reversed, the winding and discharging of the operation wires are reversed. Accordingly, the bending direction of the bending section is also reversed. In this way, the bending section can bend left-and-right directions or up- and down directions. If two pulleys which have the above-described configuration and which can rotate independently from each other are provided in the bending operation transmission mechanism, the bending section can bend both the left-and-right directions and the up- and down directions. The bending section can bend in any direction by combining these curving directions.

Jpn. Pat. Appln. KOKAI Publication No. 2001-252244 and Jpn. Pat, Appln. KOKAI Publication No. 2002-291686 disclose an endoscope sending device in which an operation wire having its distal end connected to a bending section is coupled in an operation section to a relay wire having its proximal end fixed to a pulley. In this endoscope bending device, a mechanism configured to absorb the slack of the operation wire is provided in a coupling portion between the operation wire and the relay wire.

In another endoscope bending device, an operation wire is fixed to a pulley via a chain. In this bending device, the chain to which the proximal end of operation wire is coupled is folded, and the slack of the operation wire is thereby absorbed.

### BRIEF SUMMARY OF THE INVENTION

According to one aspect of the invention, an endoscope bending device includes: an endoscope insertion section which includes a bending section configured to bend; a bending operation section which is provided to a proximal side of the endoscope insertion section, and which is configured to perform a bending operation of the bending section; a pulley which includes a rotary portion configured to be rotated in a first rotation direction and in a second rotation direction opposite to the first rotation direction by the bending operation in the berthing operation section, an outer peripheral groove provided in an outer peripheral surface along circumferential directions, an inner peripheral groove provided along the circumferential directions to an inner peripheral side of the outer peripheral groove, and a relay groove communicating the outer peripheral groove with the inner peripheral groove; an operation wire which includes a wire proximal end movably provided in the inner peripheral groove of the pulley, and a wire distal end connected to the bending section, the operation wire extending into the endoscope insertion section after being wound around the outer peripheral groove or the inner peripheral groove in a neutral condition in which the bending section is not bent, the rotary portion of the pulley rotating from the neutral condition so that the operation wire is wound around the pulley or discharged from the pulley, and thereby the operation wire bending the bending section; a wire winding portion which is configured to further wind the operation wire around the outer peripheral groove or the inner peripheral groove when the operation wire is wound from the neutral condition; and a slack absorbing portion which is configured to absorb the slack of the operation wire by moving the wire proximal end of the operation wire in the inner peripheral groove in a direction opposite to a discharging direction of the operation wire during the operation of discharging the operation wire from the neutral condition.

Advantages of the invention will be set forth in the description which follows, and in part will be obvious from the description, or may be learned by practice of the invention. The advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1 is a schematic diagram of an endoscope according to a first embodiment of the present invention;

FIG. 2 is a perspective view showing a bending device according to the first embodiment;

FIG. 3 is a sectional view showing the bending device according to the first embodiment;

FIG. 4 is a sectional view showing a first rotary cylindrical portion and a first pulley of the bending device according to the first embodiment;

FIG. 5A is a sectional view along the line 5A-5A of FIG. 4 in a neutral condition in which a bending section is not bent;

FIG. 5B is a sectional view along the line 5B-5B of FIG. 4 in the neutral condition in which the bending section is not bent;

FIG. 6A is a sectional view of the position along the line 5A-5A of FIG. 4 showing a condition in which the first pulley is rotated from the condition of FIG. 5A in a counterclockwise direction when viewed from above in FIG. 4;

FIG. 6B is a sectional view of the position along the line 5B-5B of FIG. 4 showing the condition in which the first pulley is rotated from the condition of FIG. 5B in the counterclockwise direction when viewed from above in FIG. 4;

FIG. 7A is a sectional view of the position along the line 5A-5A of FIG. 4 showing a condition in which the first pulley is rotated from the condition of FIG. 5A in a clockwise direction when viewed from above in FIG. 4;

FIG. 7B is a sectional view of the position along the line 5B-5B of FIG. 4 showing the condition in which the first pulley is rotated from the condition of FIG. 5B in the clockwise direction when viewed from above in FIG. 4;

FIG. 8 is a sectional view showing a first rotary cylindrical portion and a first pulley of a bending device according to a second embodiment of the present invention;

FIG. 9A is a sectional view along the line 9A-9A of FIG. 8 in a neutral condition in which a bending section is not bent;

FIG. 9B is a sectional view along the line 9B-9B of FIG. 8 in the neutral condition in which the bending section is not bent;

FIG. 10A is a sectional view of the position along the line 9A-9A of FIG. 8 showing a condition in which an intermediate disk is rotated from the condition of FIG. 9A in a counterclockwise direction when viewed from above in FIG. 8;

FIG. 10B is a sectional view of the position along the line 9B-9B of FIG. 8 showing the condition in which the intermediate disk is rotated from the condition of FIG. 9B in the counterclockwise direction when viewed from above in FIG. 8;

FIG. 11A is a sectional view of the position along the line 9A-9A of FIG. 8 showing a condition in which the intermediate disk is rotated from the condition of FIG. 9A in a clockwise direction when viewed from above in FIG. 8;

FIG. 11B is a sectional view of the position along the line 9B-9B of FIG. 8 showing the condition in which the intermediate disk is rotated from the condition of FIG. 9B in the clockwise direction when viewed from above in FIG. 8;

FIG. 12 is a sectional view showing a first rotary cylindrical portion and a first pulley of a bending device according to a third embodiment of the present invention;

FIG. 13A is a sectional view along the line 13A-13A of FIG. 12 in a neutral condition in which a bending section is not bent;

FIG. 13B is a sectional view along the line 13B-13B of FIG. 12 in the neutral condition in which the bending section is not bent;

FIG. 14A is a sectional view of the position along the line 13A-13A of FIG. 12 showing a condition in which an intermediate disk is rotated from the condition of FIG. 13A in a counterclockwise direction when viewed from above in FIG. 12;

FIG. 14B is a sectional view of the position along the line 13B-13B of FIG. 12 showing the condition in which the intermediate disk is rotated from the condition of FIG. 13B in the counterclockwise direction when viewed from above in FIG. 12;

FIG. 15A is a sectional view of the position along the line 13A-13A of FIG. 12 showing a condition in which the intermediate disk is rotated from the condition of FIG. 13A in a clockwise direction when viewed from above in FIG. 12;

FIG. 15B is a sectional view of the position along the line 13B-13B of FIG. 12 showing the condition in which the intermediate disk is rotated from the condition of FIG. 13B in the clockwise direction when viewed from above in FIG. 12;

FIG. 16 is a sectional view showing a first rotary cylindrical portion and a first pulley of a bending device according to a fourth embodiment of the present invention;

FIG. 17A is a sectional view along the line 17A-17A of FIG. 16 in a neutral condition in which a bending section is not bent;

FIG. 17B is a sectional view along the line 17B-17B of FIG. 16 in the neutral condition in which the bending section is not bent;

FIG. 18 is a plan view showing a second pulley component of the first pulley according to the third embodiment;

FIG. 19A is a sectional view of the position along the line 17A-17A of FIG. 16 showing a condition in which a first pulley component is rotated from the condition of FIG. 17A in a counterclockwise direction when viewed from above in FIG. 16;

FIG. 19B is a sectional view of the position along the line 17B-17B of FIG. 16 showing the condition in which the first pulley component is rotated from the condition of FIG. 17B in the counterclockwise direction when viewed from above in FIG. 16;

FIG. 20A is a sectional view of the position along the line 17A-17A of FIG. 16 showing a condition in which the first pulley component is rotated from the condition of FIG. 17A in a clockwise direction when viewed from above in FIG. 16; and

FIG. 20B is a sectional view of the position along the line 17B-17B of FIG. 16 showing the condition in which the first pulley component is rotated from the condition of FIG. 17B in the clockwise direction when viewed from above in FIG. 16.

### DETAILED DESCRIPTION OF THE INVENTION

### First Embodiment

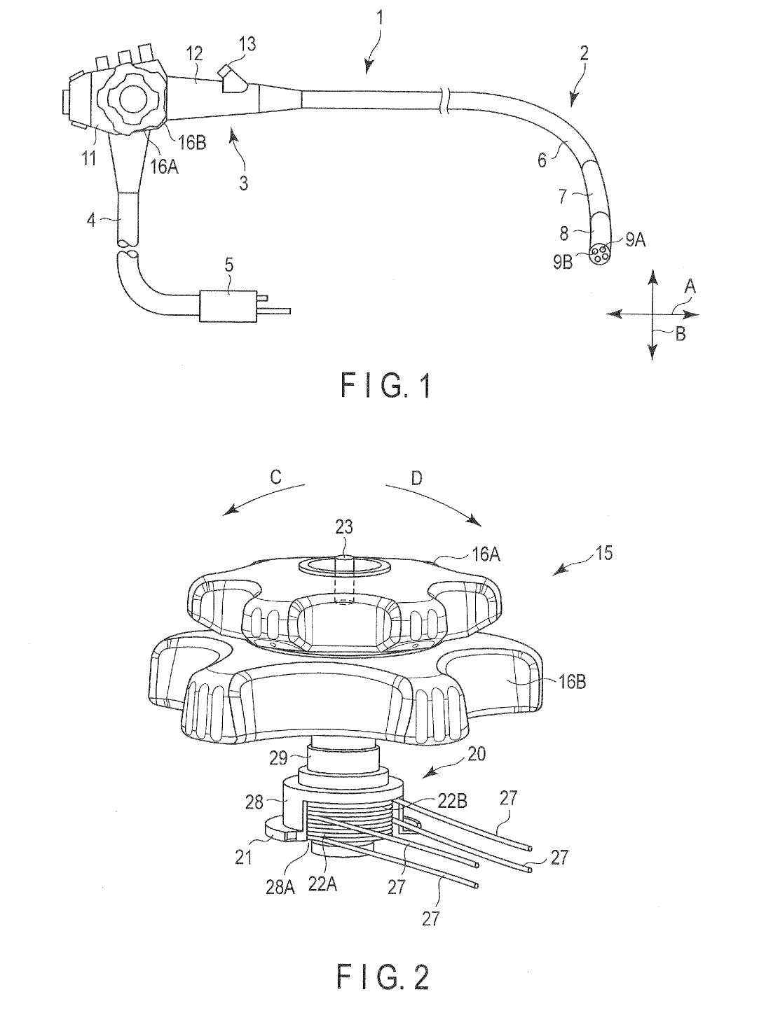

A first embodiment of the present invention is described with reference to FIG. 1 to FIG. 7B.

FIG. 1 is a diagram showing the configuration of an endoscope 1. The endoscope 1 includes an elongate insertion section 2 configured to be inserted into a body cavity, and an operation section 3 coupled to a proximal side of the insertion section 2. One end of a universal cord 4 is connected to the operation section 3. The other end of the universal cord 4 is connected to, for example, an image observation unit and an illumination power supply unit (none of which are shown) via a scope connector 5.

The insertion section 2 includes an elongate flexible section 6 having flexibility, a bending section 7 coupled to a distal side of the flexible section 6, and a distal rigid section 9 provided to the distal side of the bending section 7. The bending section 7 is configured to bend left-and-right directions (the directions of an arrow A in FIG. 1) and up-and-down directions (the directions of an arrow B in FIG. 1). The bending section 7 can bend in any direction by combining the above bending directions.

For example, an observation window 9A and an illumination window 9B are provided at a distal face of the distal rigid section 8. In the distal rigid section 8, an image pickup element (not shown) is provided to face the observation window 9A. The image pickup element is configured to image a subject via the observation window 9A. An imaging cable (not shown) is connected to the image pickup element. The imaging cable extends to the scope connector 5 through the insertion section 2, the operation section 3, and the universal cord 4. In the insertion section 2, a light guide (not shown) is provided to guide light illuminating the subject to illumination window 9B. The light guide extends to the scope connector 5 through the operation section 3 and the universal cord 4.

The operation section 3 includes an operation section casing 11, and a holding section casing 12 provided to a side where the insertion section 2 is located of the operation section casing 11. The holding section casing 12 is provided with a forceps opening 13.

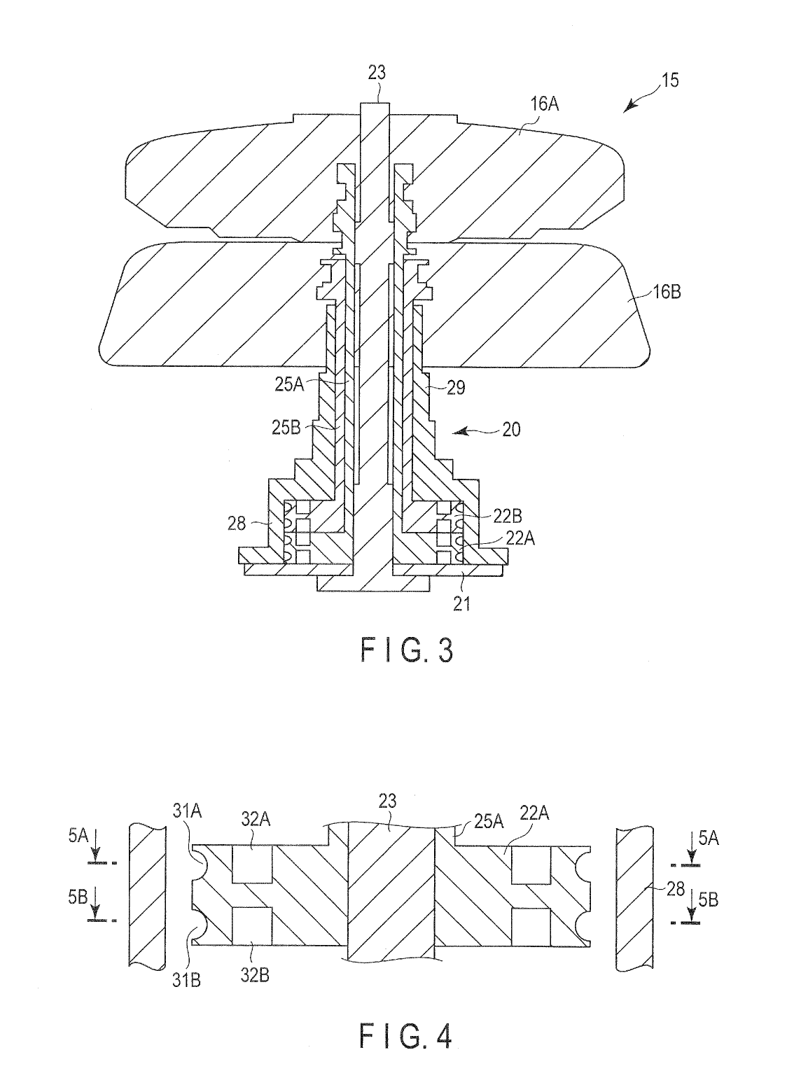

FIG. 2 and FIG. 3 are diagrams showing a bending device 15 configured to bend the bending section 7. As shown in FIG. 2, the bending device 15 includes a first bending operation knob 16A and a second bending operation knob 16E, which are bending operation sections provided in the operation section casing 11 of the operation section 3 (see FIG. 1). The bending section 7 is bent the left-and-right directions by rotating the first bending operation knob 16A. The bending section 7 is bent the up-and-down directions by rotating the second bending operation knob 16B. The first bending operation knob 16A and the second bending operation knob 16B are coupled to a bending operation transmission mechanism 20 provided in the operation section 3.

As shown in FIG. 3, the bending operation transmission mechanism 20 is fixed to a substrate 2 in the operation section 3. The substrate 21 is fixed to the inner bottom of the operation section casing 11, for example, via a screw (not shown). The bending operation transmission mechanism 20 includes a first pulley 22A used to bend the bending section 7 in the left-and-right directions, and a second pulley 22B used to bend the bending section 7 in the up-and-down directions. The first pulley 22A is provided on the upper surface of the substrate 21. The second pulley 22B is provided above the first pulley 22A. The first pulley 22A and the second pulley 22B are located substantially coaxially with the first bending operation knob 16A and the second bending operation knob 16B.

A lower end of a shaft member 23 passing through axial centers of the first pulley 22A and the second pulley 22B is fixed to the substrate 21. An upper end of the shaft member 23 passes through the second bending operation knob 16B and the first bending operation knob 16A.

A first rotary cylindrical portion 25A which is a rotation transmitting portion formed integrally with the first pulley 22A is provided outside the shaft member 23. The first rotary cylindrical portion 25A is rotatable relative to the shaft member 23. An upper end of the first rotary cylindrical portion 25A is coupled to the first bending operation knob 16A. The first rotary cylindrical portion 25A and the first pulley 22A rotate around the shaft member 23 by rotating the first bending operation knob 16A. That is, the first pulley 22A is a rotary portion configured to be rotated by the bending operation in the first bending operation knob 16A.

A second rotary cylindrical portion 25B which is a rotation transmitting portion formed integrally with the second pulley 22B is provided outside the first rotary cylindrical portion 25A. The second rotary cylindrical portion 25B is rotatable relative to the shaft member 23 independently from the first rotary cylindrical portion 25A. An upper end of the second rotary cylindrical portion 25B is coupled to the second bending operation knob 16B. The second rotary cylindrical portion 25B and the second pulley 22B rotate around the shaft member 23 by rotating the second bending operation knob 16B. That is, the second pulley 22B is a rotary portion configured to be rotated by the bending operation in the second bending operation knob 16B.

Wire proximal ends 37 of two (a pair of) operation wires 27 are connected to the first pulley 22A and the second pulley 22B, respectively. A distal end of each of the operation wires 27 is fixed to a distal end of the bending section 7 through the flexible section 6. When the first pulley 22A is rotated, one of the pair of operation wires 27 connected to the first pulley 22A is wound around the first pulley 22A, and the other operation wire is discharged from the first pulley 22A. As a result, the bending section 7 bends left-and-right directions. Similarly, when the second pulley 22B is rotated, one of the pair of operation wires 27 connected to the second pulley 22B is wound around the second pulley 22B, and the other operation wire is discharged from the second pulley 22B. As a result, the bending section 7 bends up-and-down directions.

A cylindrical guide portion 28 configured to prevent, the deviation of the operation wires 27 is provided outside the first pulley 22A and the second pulley 22B. A lower end of the guide portion 28 fixed to the substrate 21, for example, by a screw (not shown). An opening 28A is formed in the peripheral wall of the guide portion 28. The operation wires 27 extend into the insertion portion 2 from the first pulley 22A and the second pulley 22B through the opening 28A. A cylindrical portion 29 is formed integrally with the guide portion 28 above the guide portion 28. The first rotary cylindrical, portion 25A and the second rotary cylindrical portion 25B are inserted in the cylindrical portion 29.

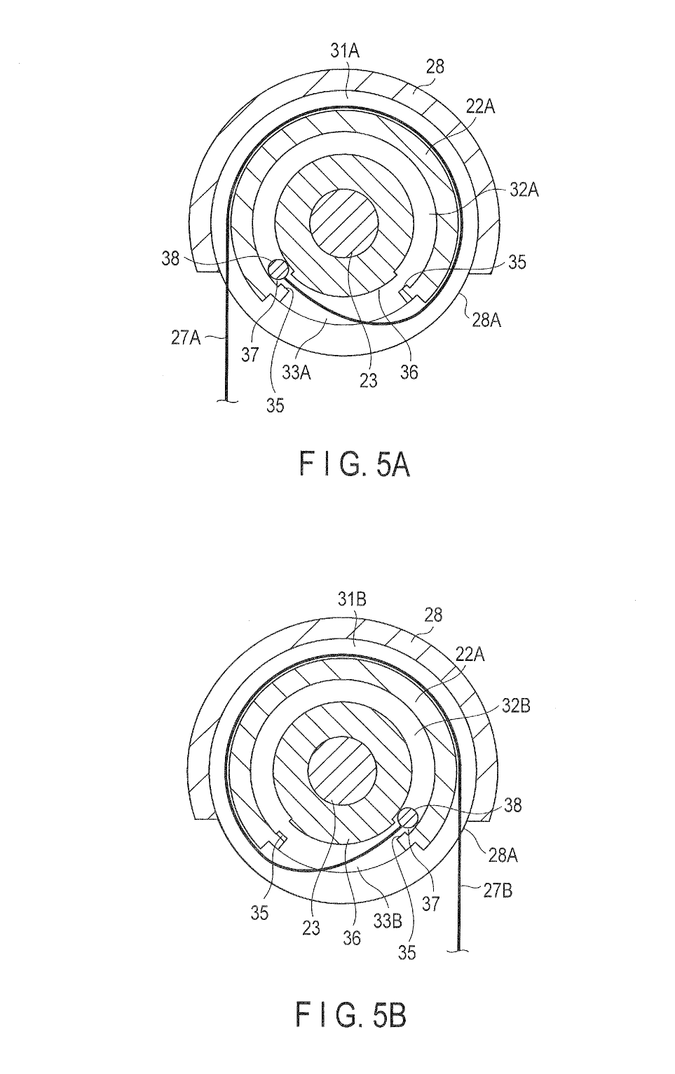

FIG. 4 to FIG. 5B are diagrams showing the configurations of the first pulley 22A and the first rotary cylindrical portion 25A. Although the first pulley 22A and the first rotary cylindrical portion 25A are described below, the same applies to the second pulley 22B and the second rotary cylindrical portion 25B.

As shown in FIG. 4, a first outer peripheral groove 31A and a second outer peripheral groove 31B which are two ring-shaped outer peripheral grooves are provided axially side by side in an outer peripheral surface of the first pulley 22A. The first outer peripheral groove 31A and the second outer peripheral groove 31B are provided separately from each other in axial directions of the first pulley 22A. Along the circumferential directions of the first pulley 22A, first inner peripheral groove 32A is provided in an upper surface of the first pulley 22A, and a second inner peripheral groove 32B is provided in a lower surface of the first pulley 22A. The first inner peripheral groove 32A and the second inner peripheral groove 32B are provided separately from each other in the axial directions of the first pulley 22A. The second inner peripheral groove 32B is located to an inner peripheral side of the first outer peripheral groove 31A, and the second inner peripheral groove 32B is located to the inner peripheral side of the second cuter peripheral groove 31B. As shown in FIG. 5A and FIG. 5B, the first pulley 22A includes a first relay groove 33A which communicates the first outer peripheral groove 31A with the first inner peripheral groove 32A, and a second relay groove 33B which communicates the second outer peripheral groove 31B with the second inner peripheral groove 32B. In a neutral condition in which the bending section 7 is not bent, the first relay groove 33A and the second relay groove 33B are located substantially in phase in the circumferential directions of the first pulley 22A. Projections 35 protruding toward the inner peripheral side from an outer peripheral wall of the first inner peripheral groove 32A is provided at both ends of the first relay groove 33A in the circumferential direction of the first pulley 22A. A projection 36 protruding toward an outer peripheral side from an inner peripheral wall of the first inner peripheral groove 32A is formed in the first relay groove 33A. As is the case with the first relay groove 33A, the second relay groove 33B is provided with projections 35 and a projection 36.

The wire proximal end 37 of a first operation wire 27A which is one of the pair of operation wires 27 connected to the first pulley 22A is located in the first inner peripheral groove 32A. A columnar press-bonded element 38 is press-bonded to the wire proximal end 37 of the first operation wire 27A. The wire proximal end 37 of the first operation wire 27A is movable in the first inner peripheral groove 32A. If the wire proximal end 37 of the first operation wire 27A moves to an end of the first inner peripheral groove 32A in the circumferential directions of the first pulley 22A, the press-bonded element 38 collides with the projection 35 and the projection 36. As a result, the movement, of the wire proximal end 37 to the first relay groove 33A is regulated.

FIG. 5A shows a neutral condition in which the bending section 7 is not bent. In the neutral condition, the wire proximal end 37 of the first operation wire 27A is located at the end of the first inner peripheral groove 32A on a left side of the first relay groove 33A in FIG. 5A. The first operation wire 27A is wound around the first outer peripheral groove 31A through the first relay groove 33A only one time in a counterclockwise direction when viewed from above in FIG. 4. The first operation wire 27A then extends into the insertion section 2 from the first outer peripheral groove 31A.

The wire proximal end 37 of a second operation wire 27B which is the other of the pair of operation wires 27 connected to the first pulley 22A is movably located in the second inner peripheral groove 32B. The press-bonded element 38 is press-bonded to the wire proximal end 37 of the second operation wire 27B as in the first operation wire 27A. If the wire proximal end 37 moves to an end of the second inner peripheral groove 32B in the circumferential directions of the first pulley 22A, the press-bonded element 38 collides with the projection 35 and the projection 36. As a result, the movement of the wire proximal end 37 to the second relay groove 33B is regulated.

FIG. 5B shows the neutral condition in which the bending section 7 is not bent. In the neutral condition, the wire proximal end 37 of the second operation wire 27B is located at the end of the second inner peripheral groove 32B on a right side of the second relay groove 33B in FIG. 59. The second operation wire 27B is wound around the second outer peripheral groove 31B through the second relay groove 33B only one time in a clockwise direction when viewed from above in FIG. 4. The second operation wire 27B then extends into the insertion section 2 from the second outer peripheral groove 31B.

Now, the function of the bending device 15 according to the present embodiment is described. Although only the case where the bending section 7 is bent in the left-and-right directions by the bending operation in the first bending operation knob 16A is described below, the same applies to the case where the bending section 7 is bent in the up-and-down directions by the bending operation in the second bending operation knob 16B.

In order to bend the bending section 7 in the left- and right directions, an operator rotates the first bending operation knob 16A, for example, in a direction of an arrow C in FIG. 2. Thus, the first rotary cylindrical portion 25 and the first pulley 22A rotate in the counterclockwise direction (first rotation direction) when viewed from above in FIG. 4.

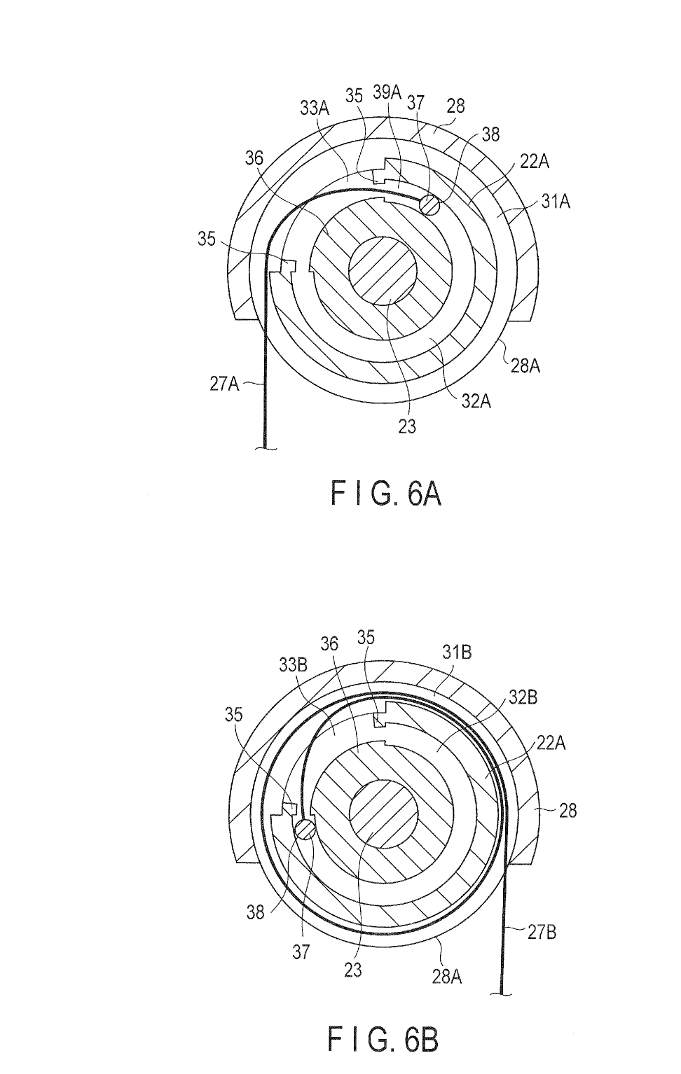

FIG. 6A and FIG. GB show a conditions in which the first pulley 22A is rotated from the neutral condition in the counterclockwise direction when viewed from above in FIG. 4. As shown in FIG. 6B, when the first pulley 22A is rotated counterclockwise, the second outer peripheral groove 31B rotates counterclockwise, and the second operation wire 27B is wound around the second cuter peripheral groove 31B. That is, when the second operation wire 27B is wound from the neutral condition, the second cuter peripheral groove 31B serves as a wire winding portion (second wire winding portion) around which the second operation wire 27B is wound. In this case, the second operation wire 27B is wound around the second outer peripheral groove 31B twice.

On the other hand, as shown in FIG. GA, when the first pulley 22A is rotated counterclockwise, a part of the first operation wire 27A wound around the first outer peripheral groove 31A in the neutral condition is discharged. In this case, the wire proximal end 37 of the first operation wire 27A is movable in the first inner peripheral groove 32A. Therefore, when the first operation wire 27A is slack, the wire proximal end 37 moves in the first inner peripheral groove 32A in a direction opposite to a discharging direction of the first operation wire 27A. As a result, the slack of the first operation wire 27A is absorbed. That is, the first inner peripheral groove 32A is provided with a slack absorbing portion (first slack absorbing portion) 39A configured to absorb the slack of the first operation wire 27A when the first operation wire 27A is discharged from the first outer peripheral groove 31A.

In this way, the first operation wire 27A is discharged and the second operation wire 27B is wound from the neutral condition, so that the bending section 7 bends in a predetermined direction (e.g. right direction).

In order to bend the bending section 7 in an opposite direction (e.g. left direction), the operator rotates the first bending operation knob 16A in a direction of an arrow D in FIG. 2. Thus, the first rotary cylindrical portion 25A and the first pulley 22A rotate in the clockwise direction (second rotation direction) when viewed from above in FIG. 4.

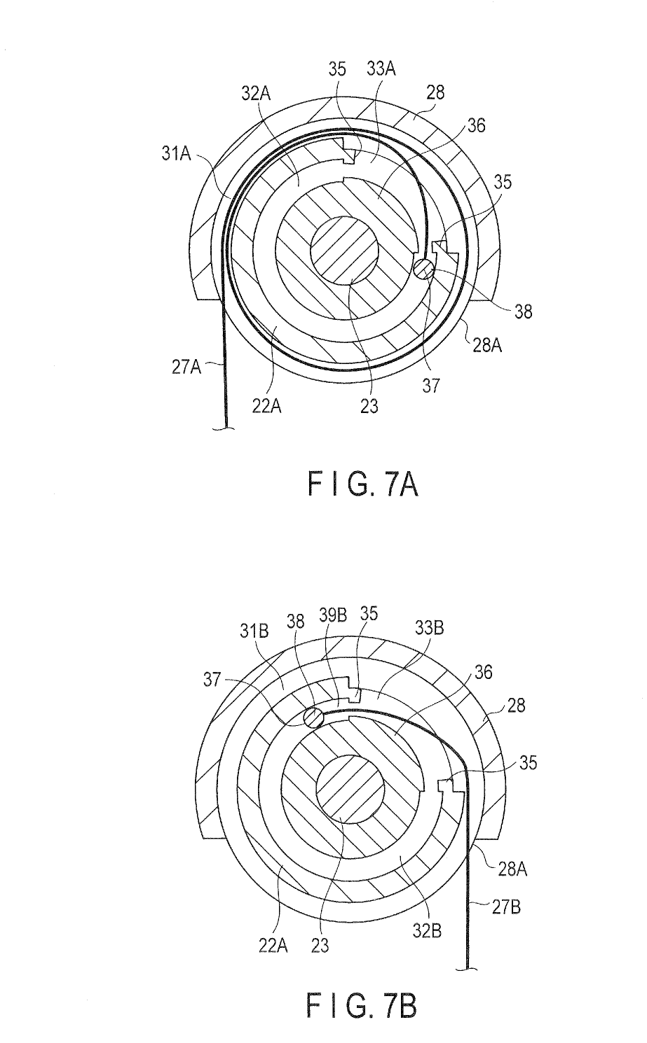

FIG. 7A and FIG. 75 show a conditions in which the first pulley 22A is rotated from the neutral condition in the clockwise direction when viewed from above in FIG. 4. As shown in FIG. 7A, when the first pulley 22A is rotated clockwise, the first outer peripheral groove 31A rotates clockwise, and the first operation wire 27A is wound around the first outer peripheral groove 31A. That is, when the first operation wire 27A is wound from the neutral condition, the first outer peripheral groove 31A serves as a wire winding portion (first wire winding portion) around which the first operation wire 27A is wound. In this case, the first operation wire 27A is wound around the first outer peripheral groove 31A twice.

On the other hand, as shown in FIG. 75, when the first pulley 22A is rotated clockwise, a part of the second operation wire 27B wound around the second outer peripheral groove 31B in the neutral condition is discharged. In this case, the wire proximal end 37 of the second operation wire 27B is movable in the second inner peripheral groove 32B. Therefore, when the second operation wire 27B is slack, the wire proximal end 37 moves in the second inner peripheral groove 32B in a direction opposite to a discharging direction of the second operation wire 27B. As a result, the slack of the second operation wire 27B is absorbed. That is, the second inner peripheral groove 32B is provided with a slack absorbing portion 39B (second slack absorbing portion) configured to absorb the slack of the second operation wire 27B when the second operation wire 27B is discharged from the second outer peripheral groove 31B.

Thus, the bending device 15 having the above-described configuration provides the following advantageous effects. That is, the first pulley 22A and the second pulley 22B of the bending device 15 include the first inner peripheral groove 32A provided in the upper surface and the second inner peripheral groove 32B provided in the lower surface along the circumferential directions. The wire proximal end 37 of the first operation wire 27A is movable in the first inner peripheral groove 32A, and the wire proximal end 37 of the second operation wire 27B is movable in the second inner peripheral groove 32B. If the first pulley 22A rotates from the neutral condition in which the bending section 7 is not bent, one of the first operation wire 27A and the second operation wire 27B is discharged from the first pulley 22A, and the other is wound around the first pulley 22A. The same applies to the rotation of the second pulley 22B from the neutral condition. For example, when the first operation wire 27A is discharged, the first operation wire 27A may be slack. In this case, the wire proximal end 37 of the first operation wire 27A moves in the first inner peripheral groove 32A in the direction opposite to the discharging direction of the first operation wire 27A. As a result, the slack of the first operation wire 27A is absorbed. When the second operation wire 27B is discharged, the slack of the second operation wire 27B is absorbed in the same manner. As described above, in the bending device 15, the first pulley 22A and the second pulley 22B are provided with spaces configured to absorb the slack of the operation wire 27. Thus, the slack of the operation wire 27 can be effectively absorbed without being affected by the design limitation of the operation section 3.

The first inner peripheral groove 32A and the second inner peripheral groove 32B are formed along the circumferential directions of the first pulley 22A and the second pulley 22B. This makes it possible to secure sufficient space (length) to absorb the slack of the operation wire 27. Thus, the slack of the long operation wire 27 can be sufficiently absorbed, which is advantageous in size reduction of the operation section 3.

### Modification of First Embodiment

In the embodiment described above, both ends of the first inner peripheral groove 32A communicate with the first outer peripheral groove 31A via the first relay groove 33A, and both ends of the second inner peripheral groove 32B communicate with the second outer peripheral groove 31B via the second relay groove 33B. However, the present invention is not limited thereto. For example, only one end of the first inner peripheral groove 32A may communicate with the first outer peripheral groove 31A.

Furthermore, in the embodiment described above, the first operation wire 27A is wound in the counterclockwise direction when viewed from above in FIG. 4, and the second operation wire 27B is wound in the clockwise direction when viewed from above in FIG. 4. However, the present invention is not limited thereto. That is, the second operation wire 27B has only to be configured to be wound in a direction opposite to a winding direction of the first operation wire 27A.

Still further, in the embodiment described above, the movement of the wire proximal end 37 of the first operation wire 27A to the first relay groove 33A and the movement of the wire proximal end 37 of the second operation wire 27B to the second relay groove 33B are regulated by the collisions of the press-bonded elements 38 with the projections 35 and the projections 36. However, the present invention is not limited thereto. That is, the movement of the wire proximal end 37 of the first operation wire 27A to the first relay groove 33A and the movement of the wire proximal end 37 of the second operation wire 27B to the second relay groove 33B have only to be configured to be regulated.

### Second Embodiment

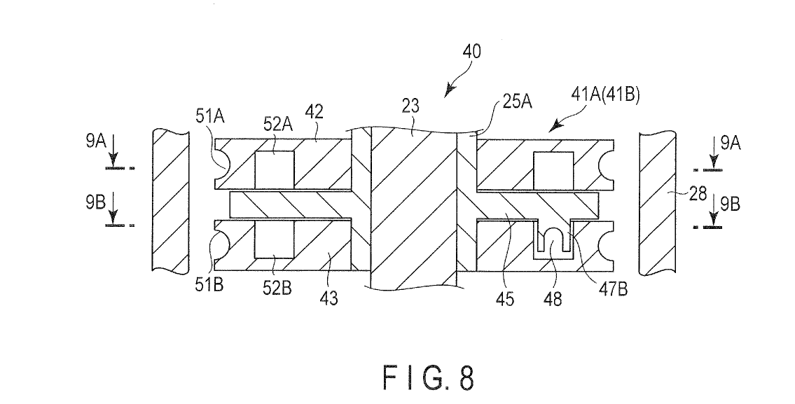

Now, a second embodiment of the present invention is described with reference to FIG. 8 to FIG. 11B. In the second embodiment, the configuration of the bending device 15 according to the first embodiment is modified as below. Components having the same parts and the same functions as those in the first embodiment are provided with the same reference signs and are not described.

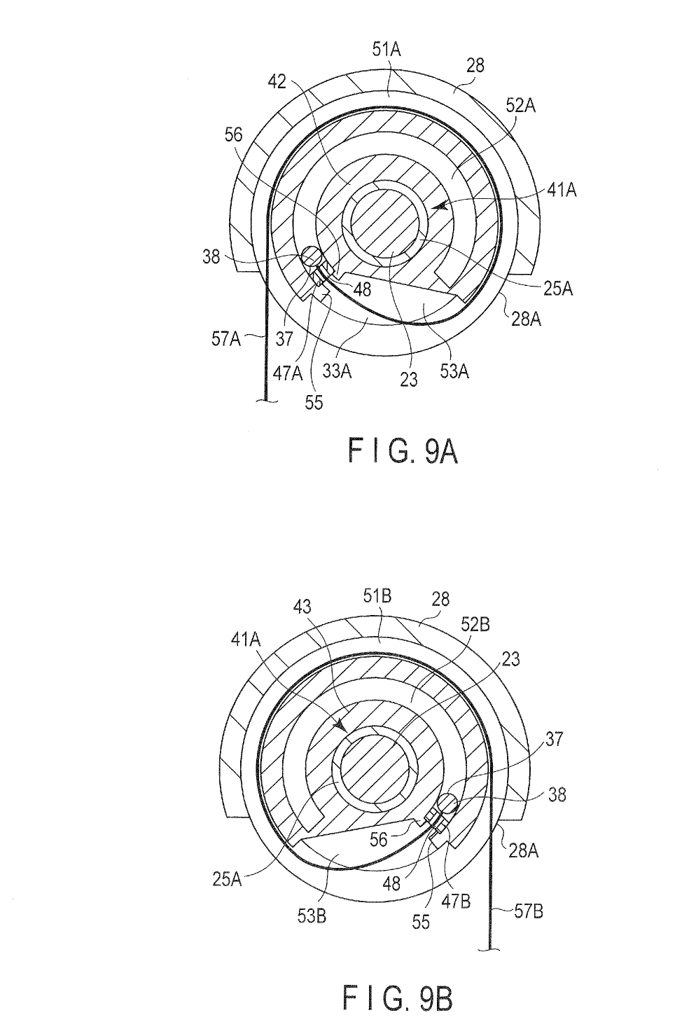

FIG. 8 to FIG. 9B are diagrams showing the configurations of a first pulley 41A and a first rotary cylindrical portion 25A of a bending device 40 according to the present embodiment. Although the first pulley 41A and the first rotary cylindrical portion 25A are described below, the same applies to a second pulley 41B and a second rotary cylindrical portion 25B.

As shown in FIG. 8 to FIG. 9B, the first pulley 41A includes a substantially columnar first pulley component 42 provided on an upper side in axial directions, and a substantially columnar second pulley component 43 provided on a lower side in the axial directions. An intermediate disk 45 formed integrally with the first rotary cylindrical portion 25A is provided between the first pulley component 42 and the second pulley component 43. The intermediate disk 45 can rotate in directions around the axis of the first pulley 41A together with the first rotary cylindrical portion 25A which is d rotation transmitting portion. That is, the intermediate disk 45 is a rotary portion which is rotated by the bending operation in a first bending operation knob 16A.

Along circumferential directions of the first pulley 41A, a first outer peripheral groove 51A is formed in an outer peripheral surface of the first pulley component 42, and a second outer peripheral groove 51B is formed in an outer peripheral surface of the second pulley component 43. Moreover, along the circumferential directions of the first pulley 41A, a first inner peripheral groove 52A is provided in a lower surface of the first, pulley component 42, and a second inner peripheral groove 52B is provided in an upper surface of the second pulley component 43. The first inner peripheral groove 52A is located to an inner peripheral side of the first outer peripheral groove 51A, and the second inner peripheral groove 52B is located to the inner peripheral side of the second outer peripheral groove 51B. The first, inner peripheral groove 52A is provided between the intermediate disk 45 and the first pulley component 42, and the second inner peripheral groove 52B is provided between the intermediate disk 45 and the second pulley component 43. The first pulley component 42 is provided with first relay groove 53A which communicates the first outer peripheral groove 51A with one end of the first inner peripheral groove 52A. Similarly, the second pulley component 43 is provided with a second relay groove 53B which communicates the second outer peripheral groove 51B with one end of the second inner peripheral groove 52B. In a neutral condition in which a bending section 7 is not bent, the first relay groove 53A and the second relay groove 53B are located substantially in phase in the circumferential directions of the first pulley 41A. Moreover, in the neutral condition in which the bending section 7 is not bent, the first inner peripheral groove 52A communicates with the first outer peripheral groove 51A at its left end in FIG. 9A, and the second inner peripheral groove 52B communicates with the second outer peripheral groove 51B at its right end in FIG. 9B. At the end of the first inner peripheral groove 52A communicating with the first outer peripheral groove 51A, a pulley projection 55 protruding toward the inner peripheral side from an outer peripheral wall of the first inner peripheral groove 52A is formed, and a pulley projection 56 protruding toward an outer peripheral side from an inner peripheral wall of the first inner peripheral groove 52A is also formed. Similarly, a pulley projection 55 and a pulley projection 56 are also formed at the end of the second inner peripheral groove 52B communicating with the second outer peripheral groove 51B.

An upwardly protruding first disk projection 47A is provided on an upper surface of the intermediate disk 45, and a downwardly protruding second disk projection 47B is provided on a lower surface of the intermediate disk 45. The first disk projection 47A is movably inserted in the first inner peripheral groove 52A. The second disk projection 47B is movably inserted in the second inner peripheral groove 52B. If the first disk projection 47A moves to the end of the first inner peripheral groove 52A communicating with the first relay groove 53A, the first disk projection 47A collides with the pulley projection 55 and the pulley projection 56. As a result, the movement of the first disk projection 47A to the first relay groove 53A is regulated. Similarly, if the second disk projection 47B moves to the end of the second inner peripheral groove 52B communicating with the second relay groove 53B, the second disk projection 47B collides with the pulley projection 55 and the pulley projection 56. As a result, the movement of the second disk projection 47B to the second relay groove 53B is regulated. The first disk projection 47A and the second disk projection 47B are provided with depressions 48.

A wire proximal end 37 of a first operation wire 57A which is one of a pair of operation wires 27 connected to the first pulley 41A is located in the first inner peripheral groove 52A. A columnar press-bonded element 38 is fixed to the wire proximal end 37 of the first operation wire 57A. The wire proximal end 37 of the first operation wire 57A is movable in the first inner peripheral groove 52A.

As shown in FIG. 9A, in a neutral condition in which the bending section 7 is not bent, the wire proximal end 37 of the first operation wire 57A is located at the end of the first inner peripheral groove 52A communicating with the first outer peripheral groove 51A (the end located on the left side of the first relay groove 53A in FIG. 9A). The first operation wire 57A is inserted through the depression 48 of the first disk projection 47A. The first operation wire 57A inserted through the depression 48 is wound around the first outer peripheral groove 51A through the first relay groove 53A only one time in a counterclockwise direction when viewed from above in FIG. 8. The first operation wire 57A then extends into an insertion section 2 from the first outer peripheral groove 51A. In this case, if the wire proximal end 37 moves to the first disk projection 47A, the press-bonded element 38 collides with the first disk projection 47A. As a result, the movement of the wire proximal end 37 beyond the first disk projection 47A in a discharging direction of the first operation wire 57A is regulated. That is, the wire proximal end 37 of the first operation wire 57A in the neutral condition is located in the first inner peripheral groove 52A so that its movement in the discharging direction of the first operation wire 57A is regulated. As the movement of the first disk projection 47A to the first relay groove 53A is regulated by the pulley projection 55 and the pulley projection 56, the movement of the wire proximal end 37 to the first relay groove 53A is regulated.

If the first rotary cylindrical portion 25A and the intermediate disk 45 are rotated from the neutral condition in the counterclockwise direction (first rotation direction) when viewed from above in FIG. 8, the pulley projection 55 and the pulley projection 56 of the first pulley component 42 are pressed by the first disk projection 47A of the intermediate disk 45. Thus, the first pulley component 42 rotates counterclockwise when viewed from above in FIG. 8 together with the intermediate disk 45. In this case, the second pulley component 43 does not rotate, and the second disk projection 47B moves in the second inner peripheral groove 52B.

In the second inner peripheral groove 52B, a wire proximal end 37 of a second operation wire 57B which is the other of the pair of operation wires 27 connected to the first pulley 41A is moveable. A press-bonded element 38 is fixed to the wire proximal end 37 of the second operation wire 57B as in the first operation wire 57A.

As shown in FIG. 9B, in the neutral condition in which the bending section 7 is not bent, the wire proximal end 37 of the second operation wire 57B is located at the end of the second inner peripheral groove 52B communicating with the second outer peripheral groove 51B (the end located on the right side of the second relay groove 53B in FIG. 9B). The second operation wire 57B is inserted through the depression 48 of the second disk projection 47B. The second operation wire 57B inserted through the depression 48 is wound around the second outer peripheral groove 51B through the second relay groove 53B only one time in a clockwise direction when viewed from above in FIG. 8. The second operation wire 57B then extends into the insertion section 2 from the second outer peripheral groove 51B. In this case, if the wire proximal end 37 moves to the second disk projection 47B, the press-bonded element 38 collides with the second disk projection 47B. As a result, the movement of the wire proximal end 37 beyond the second disk projection 47B in a discharging direction of the second operation wire 57B is regulated. That is, the wire proximal end 37 of the second operation wire 57B in the neutral condition is located in the second inner peripheral groove 52B so that its movement in the discharging direction of the second operation wire 57B is regulated. As the movement of the second disk projection 47B to the second relay groove 53B is regulated by the pulley projection 55 and the pulley projection 56, the movement of the wire proximal end 37 to the second relay groove 53B is regulated.

If the first rotary cylindrical portion 25A and the intermediate disk 45 are rotated from the neutral condition in the clockwise direction (second rotation direction) when viewed from above in FIG. 8, the pulley projection 55 and the pulley projection 56 of the second pulley component 43 are pressed by the second disk projection 47B of the intermediate disk 45. Thus, the second pulley component 43 rotates clockwise when viewed from above in FIG. 8 together with the intermediate disk 45. In this case, the first pulley component 42 does not rotate, and the first disk projection 47A moves in the first inner peripheral groove 52A.

Now, the function of the bending device 40 according to the present embodiment is described. Although only the case where the bending section 7 is bent in the left-and-right directions by the first bending operation knob 16A is described below, the same applies to the case where the bending section 7 is bent in the up-and-down directions by a second bending operation knob 16B.

In order to bend the bending section in the left-and-right directions 7, the operator rotates the first bending operation knob 16A, for example, in the direction of the arrow C in FIG. 2. Thus, the first rotary cylindrical portion 25A and the intermediate disk 45 of the first pulley 41A rotate in the counterclockwise direction (first rotation direction) when viewed from above in FIG. 8.

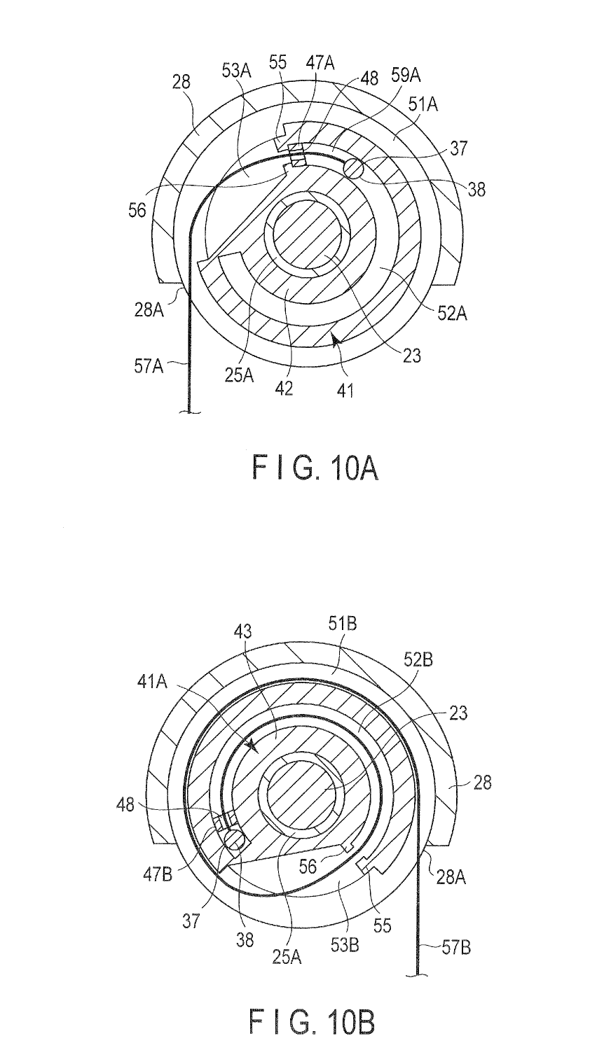

FIG. 10A and FIG. 10B show a conditions in which the intermediate disk 45 of the first pulley 41A is rotated from the neutral condition in the counterclockwise direction when viewed from above in FIG. 8. As shown in FIG. 10B, when the intermediate disk 45 of the first pulley 41A is rotated counterclockwise when viewed from above in FIG. 8, the second disk projection 47B of the intermediate disk 45 moves in the second inner peripheral groove 52B of the second pulley component 43 in a direction opposite to the discharging direction of the second operation wire 57B. In this case, the second pulley component 43 does not rotate. The movement of the wire proximal end 37 beyond the second disk projection 47B in the discharging direction of the second operation wire 57B is regulated. Thus, in response to the movement of the second disk projection 47B, the wire proximal end 37 of the second operation wire 57B moves in the second inner peripheral groove 52B in the direction opposite to the discharging direction of the second operation wire 57B together with the second disk projection 47B. As a result, the second operation wire 57B is wound around the second inner peripheral groove 52B. That is, when the second operation wire 57B is wound from the neutral condition, the second inner peripheral groove 52B serves as a wire winding portion (second wire winding portion) around which the second operation wire 57B is wound. In this case, the second operation wire 57B is wound around the second inner peripheral groove 52B, and wound around the second outer peripheral groove 51B through the second relay groove 53B only one time. The second operation wire 57B then extends into the insertion section 2. Therefore, the second operation wire 57B is not wound around the second outer peripheral groove 51B twice.

On the other hand, as shown in FIG. 10A, when the intermediate disk 45 of the first pulley 41A is rotated counterclockwise, the pulley projection 55 and the pulley projection 56 of the first pulley component 42 are pressed by the first disk projection 47A of the intermediate disk 45. Thus, the first pulley component 42 rotates counterclockwise when viewed from above in FIG. 8 together with the intermediate disk 45. The first pulley component 42 rotates counterclockwise so that a part of the first operation wire 57A wound around the first outer peripheral groove 51A in the neutral condition is discharged. In this case, the wire proximal end 37 of the first operation wire 57A is movable in the first inner peripheral groove 52A. Therefore, when the first operation wire 57A is slack, the wire proximal end 37 moves in the first inner peripheral groove 52A in a direction opposite to the discharging direction of the first operation wire 57A. As a result, the slack of the first operation wire 57A is absorbed. That is, the first inner peripheral groove 52A is provided with a slack absorbing portion 59A (first slack absorbing portion) configured to absorb the slack of the first operation wire 57A when the first operation wire 57A is discharged from the first outer peripheral groove 51A.

In this way, the first operation wire 57A is discharged and the second operation wire 57B is wound from the neutral condition, so that the bending section 7 bends in a predetermined direction (e.g. right direction).

In order to bend the bending section 7 in an opposite direction (e.g. left direction), the operator rotates the first bending operation knob 16A in a direction of an arrow D in FIG. 2. Thus, the first rotary cylindrical portion 25A and the intermediate disk 45 of the first pulley 41A rotate in the clockwise direction (second rotation direction) when viewed from above in FIG. 8.

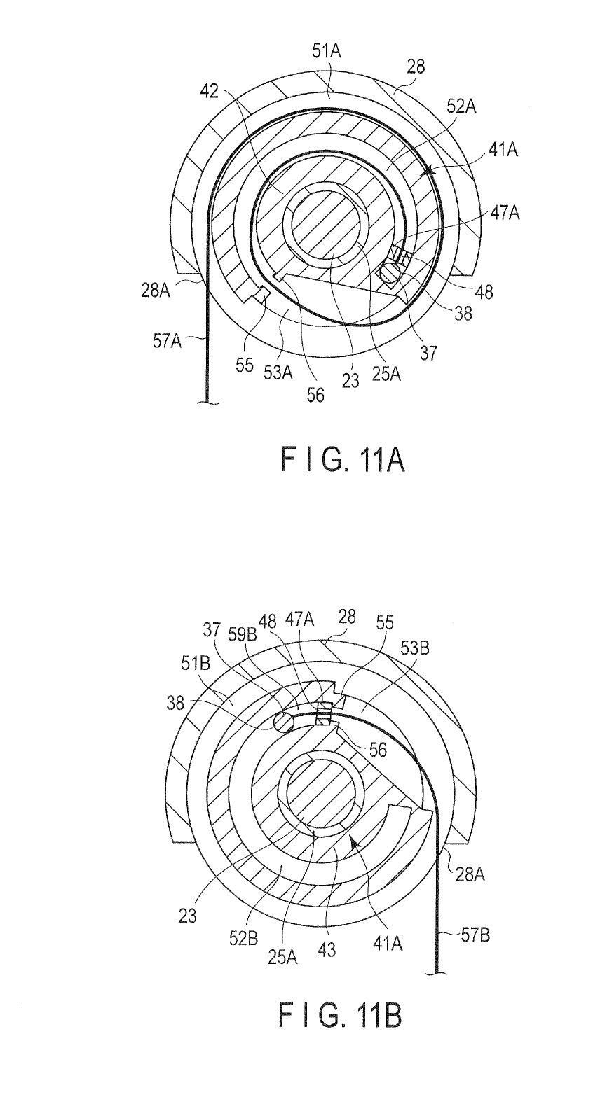

FIG. 11A and FIG. 11B show a conditions in which the intermediate disk 45 of the first pulley 41A is rotated from the neutral condition in the clockwise direction when viewed from above in FIG. 8. As shown in FIG. 11A, when the intermediate disk 45 of the first pulley 41A is rotated clockwise, the first disk projection 47A of the intermediate disk 45 moves in the first inner peripheral groove 52A of the first pulley component 42 in the direction opposite to the discharging direction of the first operation wire 57A. In this case, the first pulley component 42 does not rotate. The movement of the wire proximal end 37 beyond the first disk projection 47A in the discharging direction of the first operation wire 57A is regulated. Thus, in response to the movement of the first disk projection 47A, the wire proximal end 37 of the first operation wire 57A moves in the first inner peripheral groove 52A in the direction opposite to the discharging direction of the first operation wire 57A together with the first disk projection 47A. As a result, the first operation wire 57A is wound around the first inner peripheral groove 52A. That is, when the first operation wire 57A is wound from the neutral condition, the first inner peripheral groove 52A serves as a wire winding portion (first, wire winding portion) around which the first operation wire 57A is wound. In this case, the first operation wire 57A is wound around the first inner peripheral groove 52A, and wound around the first outer peripheral groove 51A through the first relay groove 53A only one time. The first operation wire 57A then extends into the insertion section 2. Therefore, the first operation wire 57A is not wound around the first outer peripheral groove 51A twice.

On the other hand, as shown in FIG. 11B, if the intermediate disk 45 of the first pulley 41A is rotated clockwise when viewed from above in FIG. 8, the pulley projection 55 and the pulley projection 56 of the second pulley component 43 are pressed by the second disk projection 47B of the intermediate disk 45. Thus, the second pulley component 43 rotates clockwise when viewed from above in FIG. 8 together with the intermediate disk 45. The second pulley component 43 rotates clockwise so that a part of the second operation wire 57B wound around the second outer peripheral groove 51B in the neutral condition is discharged. In this case, the wire proximal end 37 of the second operation wire 57B is movable in the second inner peripheral groove 52B. Therefore, when the second operation wire 57B is slack, the wire proximal end 37 moves in the second inner peripheral groove 52B in the direction opposite to the discharging direction of the second operation wire 57B. As a result, the slack of the second operation wire 57B is absorbed. That is, the second inner peripheral groove 52B is provided with a slack absorbing portion 59B (second slack absorbing portion) configured to absorb the slack of the second operation wire 57B when the second operation wire 57B is discharged from the second outer peripheral groove 51B.

Thus, the bending device 40 having the above-described configuration provides the following advantageous effects. That is, the first pulley 41A and the second pulley 41B include the first inner peripheral groove 52A provided in the first pulley component 42 and the second inner peripheral groove 52B provided in the second pulley component 43 along the circumferential directions. The wire proximal end 37 of the first operation wire 57A is movable in the first inner peripheral groove 52A, and the wire proximal end 37 of the second operation wire 57B is movable in the second inner peripheral groove 52B. In the first pulley 41A and the second pulley 41B, if the intermediate disk 45 rotates from the neutral condition in one of the rotation directions, one of the first pulley component 42 and the second pulley component 43 rotates together with the intermediate disk 45. Contrarily, if the intermediate disk 45 rotates from the neutral condition in the other of the rotation directions, the other of the first pulley component 42 and the second pulley component 43 rotates together with the intermediate disk 45. The first operation wire 57A is discharged by the rotation of the first pulley component 42, and the second operation wire 57B is discharged by the rotation of the second pulley component 43. For example, when the first operation wire 57A is discharged, the first operation wire 57A may be slack. In this case, the wire proximal end 37 of the first operation wire 57A moves in the first inner peripheral groove 52A in the direction opposite to the discharging direction of the first operation wire 57A. As a result, the slack of the first operation wire 57A is absorbed. When the second operation wire 57B is discharged, the slack of the second operation wire 57B is absorbed in the same manner. As described above, the first pulley 41A and the second pulley 41B of the bending device 10 are provided with the spaces configured to absorb the slack of the operation wire 27. Thus, the slack of the operation wire 27 can be effectively absorbed without being affected by the design limitation of the operation section 3.

In the bending device 40, the first inner peripheral groove 52A and the second inner peripheral groove 52B are formed along the circumferential directions of the first pulley 41A and the second pulley 41B. This makes it possible to secure sufficient space (length) to absorb the slack of the operation wire 27. Thus, the slack of the long operation wire 27 can be sufficiently absorbed, which is advantageous in size reduction of the operation section 3.

Furthermore, in the bending device 40, only one of the first pulley component 42 and the second pulley component 43 rotates together with the intermediate disk 45. When the first pulley component 42 does not rotate together with the intermediate disk 45, the first disk projection 47A moves in the first inner peripheral groove 52A the direction opposite to the discharging direction of the first operation wire 57A. The movement of the wire proximal end 37 of the first operation wire 57A beyond the first disk projection 47A in the discharging direction of the first operation wire 57A is regulated. Thus, in response to the movement of the first disk projection 47A, the wire proximal end 37 of the first operation wire 57A moves in the first inner peripheral groove 52A in the direction opposite to the discharging direction of the first operation wire 57A together with the first disk projection 47A. As a result, the first operation wire 57A is wound. In this case, the first operation wire 57A is wound around the first inner peripheral groove 52A. This can prevent the first operation wire 57A from being wound around the first cuter peripheral groove 51A twice. When the second pulley component 43 does not rotate together with the intermediate disk 45, it is also possible to prevent the second operation wire 57B from being wound around the second outer peripheral groove 51B twice, too.

### Modification of Second Embodiment

In the embodiment described above, only one end of the first inner peripheral groove 52A communicates with the first outer peripheral groove 51A, and only one end of the second inner peripheral groove 52B communicates with the second outer peripheral groove 51B. However, the present invention is not limited thereto. For example, both ends of the first inner peripheral groove 52A may communicate with the first outer peripheral groove 51A. In this case, projections protruding toward the outer peripheral side from the inner peripheral wall of the first inner peripheral groove 52A are provided at both ends of the first inner peripheral groove 52A to regulate the movement of the wire proximal end 37 to the first relay groove 53A.

Furthermore, in the embodiment described above, the first operation wire 57A is wound in the counterclockwise direction when viewed from above in FIG. 8, and the second operation wire 57B is wound in the clockwise direction when viewed from above in FIG. 8. However, the present invention is not limited thereto. That is, the second operation wire 57B has only to be configured to be wound in a direction opposite to a winding direction of the first operation wire 57A.

Still further, in the embodiment described above, the movement of the wire proximal end 37 of the first operation wire 57A beyond the first disk projection 47A in the discharging direction of the first operation wire 57A is regulated by the collision of the press-bonded element 38 with the first disk projection 47A. However, the movement of the wise proximal end 37 beyond the first disk projection 47A in the discharging direction of the first operation wire 57A has only to be configured to be regulated. Similarly, the movement of the wire proximal end 37 of the second operation wire 57B beyond the second disk projection 47B in the discharging direction of the second operation wire 57B has only to be configured to be regulated. Moreover, in the neutral condition, the wire proximal end 37 of the first operation wire 57A has only to be located in the first inner peripheral groove 52A so that its movement in the discharging direction of the first operation wire 57A is regulated. Similarly, in the neutral condition, the wire proximal end 37 of the second operation wire 57B has only to be located in the second inner peripheral groove 52B so that its movement in the discharging direction of the second operation wire 57B is regulated.

Still further, in the embodiment described above, the pulley projection 55 and the pulley projection 56 of the first pulley component 42 are pressed by the first disk projection 47A of the intermediate disk 45 so that the first pulley component 42 rotates together with the intermediate disk 45. Similarly, the pulley projection 55 and the pulley projection 56 of the second pulley component 43 are pressed by the second disk projection 47B of the intermediate disk 45 so that the second pulley component 43 rotates together with the intermediate disk 45. However, one of the first pulley component 42 and the second pulley component 43 has only to be configured to rotate together with the intermediate disk 45 if the intermediate disk 45 is rotated in one of the rotation directions from the neutral condition in which the herding section 7 is not bent, and the other of the first pulley component 12 and the second pulley component 43 has to be configured to rotate together with the intermediate disk 45 if the intermediate disk 45 is rotated in the other of the rotation directions from the neutral condition.

### Third Embodiment

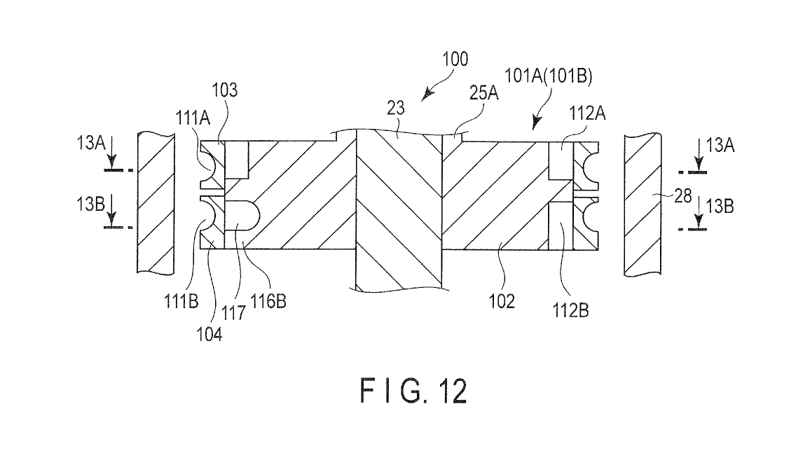

Now, a third embodiment of the present invention is described with reference to FIG. 12 to FIG. 15B. In the third embodiment, the configuration of the bending device 15 according to the first embodiment is modified as below. Components having the same parts and the same functions as those in the first embodiment are provided with the same reference signs and are not described.

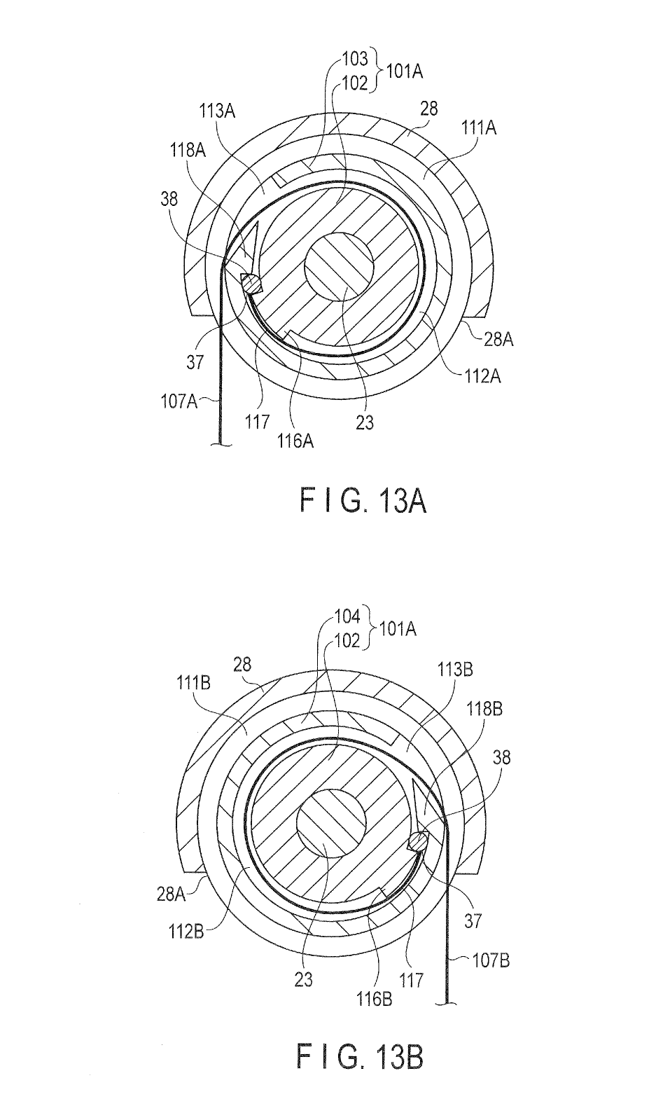

FIG. 12 to FIG. 13B are diagrams showing the configurations of a first pulley 101A and a first rotary cylindrical portion 25A of a bending device 100 according to the present embodiment. Although the first pulley 101A and the first rotary cylindrical portion 25A are described below, the same applies to a second pulley 101B and a second rotary cylindrical portion 25B.

As shown in FIG. 12 to FIG. 13B, the first pulley 101A includes a pulley body 102 that is rotatable in a directions around the axis of the first pulley 101A together with the first rotary cylindrical portion 25A which is a rotation transmitting portion. That is, the pulley body 102 is a rotary portion configured to be rotated by the bending operation in a first bending operation knob 16A. On an outer peripheral side of the pulley body 102, a substantially cylindrical first pulley component 103 is provided on an upper side in axial directions, and a substantially cylindrical second pulley component 104 is provided on a lower side in the axial directions.

Along circumferential directions of the first pulley 101A, a first outer peripheral groove 111A is formed in an outer peripheral surface of the first pulley component 103, and a second outer peripheral groove 111B is formed in an outer peripheral surface of the second pulley component 104. Moreover, a first inner peripheral groove 112A and a second inner peripheral groove 112B are provided in an outer peripheral surface of the pulley body 102 along the circumferential directions of the first pulley 101A. The first inner peripheral groove 112A is located to an inner peripheral side of the first outer peripheral groove 111A, and the second inner peripheral groove 112B is located to the inner peripheral side of the second outer peripheral groove 111B. The first inner peripheral groove 112A is provided between the pulley body 102 and the first pulley component 103, and the second inner peripheral groove 112B is provided between the pulley body 102 and the second pulley component 104. The first pulley component 103 is provided with a first relay groove 113A which communicates the first outer peripheral groove 111A with the first, inner peripheral groove 112A. The second pulley component 104 is provided with a second relay groove 113B which communicates the second outer peripheral groove 111B with the second inner peripheral groove 112B. In a neutral condition in which a bending section 7 is not bent, the first relay groove 113A and the second relay groove 113B are located out of phase with each other in the circumferential directions of the first pulley 101A. Moreover, a first component projection 118A protruding toward the inner peripheral side from an outer peripheral wall of the first inner peripheral groove 112A is formed at one end of the first inner peripheral groove 112A. Similarly, a second component projection 118B is also formed at one end of the second inner peripheral groove 112B.

The pulley body 102 is provided with a first body projection 116A and a second body projection 116B that protrude toward an outer peripheral side. The first body projection 116A is movably inserted in the first inner peripheral groove 112A. The second body projection 116B is movably inserted in the second inner peripheral groove 112B. The first body projection 116A and the second body projection 116B are provided with depressions 117.

A wire proximal end 37 of a first operation wire 107A which is one of a pair of operation wires 27 connected to the first pulley 101A is located in the first inner peripheral groove 112A. A columnar press-bonded element 38 is fixed to the wire proximal end 37 of the first operation wire 107A. The wire proximal end 37 of the first operation wire 107A is movable in the first inner peripheral groove 112A.

As shown in FIG. 13A, in a neutral condition in which the bending section 7 is not bent, the wire proximal end 37 of the first operation wire 107A is located at an end of the first inner peripheral groove 112A on a side opposite to a side where the first operation wire 107A is discharged (an end located on a lower side of the first relay groove 113A in FIG. 13A). The first operation wire 107A is inserted through the depression 117 of the first body projection 116A. The first operation wire 107A inserted through the depression 117 is wound around the first inner peripheral, groove 112A only one time in a counterclockwise direction when viewed from above in FIG. 12. The first operation wire 107A then extends into an insertion section 2 from the first outer peripheral groove 111A through the first relay groove 113A. In this case, it the wire proximal end 37 moves to the first component projection 118A, the press-bonded element 38 collides with the first component projection 118A. As a result, the movement of the wire proximal end 37 of the first operation wire 107A in a direction opposite to a discharging direction of the first operation wire 107A is regulated. That is, the wire proximal end 37 of the first operation wire 107A in the neutral condition is located in the first inner peripheral groove 112A so that its movement in the direction opposite to the discharging direction of the first operation wire 107A is regulated.

If the first rotary cylindrical portion 25A and the pulley body 102 are rotated from the neutral condition in a clockwise direction (second rotation direction) when viewed from above in FIG. 12, the first component projection 118A of the first pulley component 103 is pressed by the first body projection 116A of the pulley body 102. Thus, the first pulley component 103 rotates clockwise when viewed from above in FIG. 12 together with the pulley body 102. In this case, the second pulley component 104 does not rotate, and the second body projection 116B moves in the second inner peripheral groove 112B.

In the second inner peripheral groove 112B, a wire proximal end 37 of a second operation wire 107B which is the other of the pair of operation wires 27 connected to the first pulley 101A is moveable. A press-bonded element 38 is fixed to the wire proximal end 37 of the second operation wire 107B as in the first operation wire 107A.

As shown in FIG. 13B, in the neutral condition in which the bending section 7 is not bent, the wire proximal end 37 of the second operation wire 107B is located at an end of the second inner peripheral groove 112B on a side opposite to a side where the second operation wire 107B is discharged (an end located on a lower side of the second relay groove 113B in FIG. 13B). The second operation wire 107B is inserted through the depression 117 of the second body projection 116B. The second operation wire 107B inserted through the depression 117 is wound around the second inner peripheral groove 112B only one time in a counterclockwise direction when viewed from above in FIG. 12. The second operation wire 107B then extends into the insertion section 2 from the second outer peripheral groove 111B through the second relay groove 113B. In this case, if the wire proximal end 37 moves to the second component projection 118B, the press-bonded element 38 collides with the second component projection 118B. As a result, the movement of the wire proximal end 37 of the second operation wire 107B in a direction opposite to a discharging direction of the second operation wire 107B is regulated. That is, the wire proximal end 37 of the second operation wire 107B in the neutral condition is located in the second inner peripheral groove 112B so that its movement in the direction opposite to the discharging direction of the second operation wire 107B is regulated.

If the first rotary cylindrical portion 25A and the pulley body 102 are rotated from the neutral condition in a counterclockwise direction (first rotation direction) when viewed from above in FIG. 12, the second component projection 118B of the second pulley component 104 is pressed by the second body projection 116B of the pulley body 102. Thus, the second pulley component 104 rotates counterclockwise when viewed from above in FIG. 12 together with the pulley body 102. In this case, the first pulley component 103 does not rotate, and the first body projection 116A moves in the first inner peripheral groove 112A.

Now, the function of the bending device 100 according to the present embodiment is described. Although only the case where the bending section 7 is bent in the left-and-right directions by the first bending operation knob 16A is described below, the some applies to the case where the bending section 7 is bent in the up-and-down directions by a second bending operation knob 16B.

In order to bend the bending section 7 in the left-and-right directions, the operator rotates the first bending operation knob 16A, for example, in the direction of the arrow C in FIG. 2. Thus, the first rotary cylindrical portion 25A and the pulley body 102 of the first pulley 101A rotate in a counterclockwise direction (first rotation direction) when viewed from above in FIG. 12.

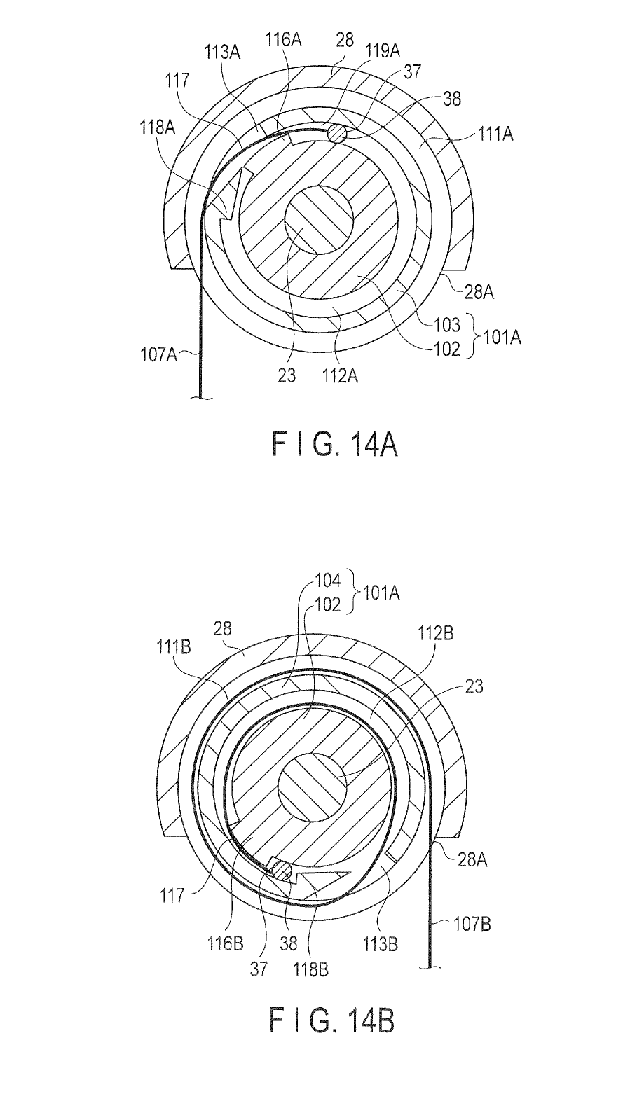

FIG. 14A and FIG. 14B show a conditions in which the pulley body 102 of the first pulley 101A is rotated from the neutral condition in the counterclockwise direction when viewed from above in FIG. 12. As shown in FIG. 14B, if the pulley body 102 of the first pulley 101A is rotated counterclockwise when viewed from above in FIG. 12, the second component projection 118B of the second pulley component 104 is pressed by the second body projection 116B of the pulley body 102. Thus, the second pulley component 104 rotates counterclockwise when viewed from above in FIG. 12 together with the pulley body 102. As the second pulley component 104 rotates counterclockwise, the second outer peripheral groove 111B rotates counterclockwise, and the second operation wire 107B is wound around the second outer peripheral groove 111B. That is, when the second operation wire 107B is wound from the neutral condition, the second outer peripheral groove 111B serves as a wire winding portion (second wire winding portion) around which the second operation wire 107B is wound. In this case, the second operation wire 107B is wound around the second inner peripheral groove 112B, and wound around the second outer peripheral groove 111B through the second relay groove 113B only one time. The second operation wire 107B then extends into the insertion section 2. Therefore, the second operation wire 107B is not wound around the second outer peripheral groove 111B twice.

On the other hand, as shown in FIG. 14A, when the pulley body 102 of the first pulley 101A is rotated counterclockwise, the first body projection 116A of the pulley body 102 moves in the first inner peripheral groove 112A in the discharging direction of the first operation wire 107A, so that the first pulley component 103 does not rotate together with the pulley body 102. In this case, in response to the rotation of the pulley body 102, the wire proximal end 37 of the first operation wire 107A moves in the first inner peripheral groove 112A in the discharging direction of the first operation wire 107A. As a result, a part of the first operation wire 107A wound around the first inner peripheral groove 112A in the neutral condition discharged. In this case, the wire proximal end 37 of the first operation wire 107A is movable in the first inner peripheral groove 112A. Therefore, when the first operation wire 107A is slack, the wire proximal end 37 moves in the first inner peripheral groove 112A in the direction opposite to the discharging direction of the first operation wire 107A. As a result, the slack of the first operation wire 107A is absorbed. That is, the first inner peripheral groove 112A is provided with a slack absorbing portion 119A (first slack absorbing portion) configured to absorb the slack of the first operation wire 107A when the first operation wire 107A is discharged from the first inner peripheral groove 112A.

In this way, the first operation wire 107A is discharged and the second operation wire 107B is wound from the neutral condition, so that the bending section 7 bends in a predetermined direction (e.g. right direction).

In order to bend the bending section 7 in an opposite direction (e.g. left direction), the operator rotates the first bending operation knob 16A in the direction of the arrow D in FIG. 2. Thus, the first rotary cylindrical portion 25A and the pulley body 102 of the first pulley 101A rotate in a clockwise direction (second rotation direction) when viewed from above in FIG. 12.

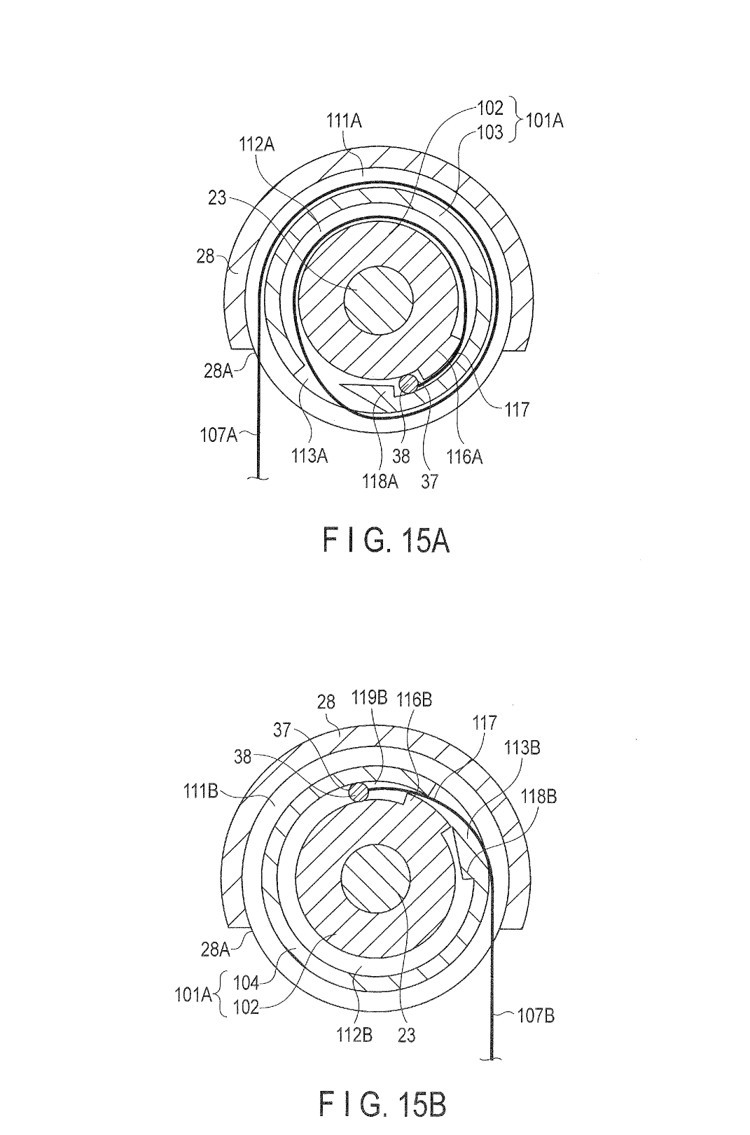

FIG. 15A and FIG. 15B show a conditions in which the pulley body 102 of the first pulley 101A is rotated from the neutral condition in the clockwise direction when viewed from above in FIG. 12. As shown in FIG. 15A, when the pulley body 102 of the first pulley 101A is rotated clockwise, the first component projection 116A of the first pulley component 103 is pressed by the first body projection 116A of the pulley body 102. Thus, the first pulley component 103 rotates clockwise when viewed from above in FIG. 12 together with the pulley body 102. As the first pulley component 103 rotates clockwise, the first outer peripheral groove 111A rotates clockwise, and the first operation wire 107A is wound around the first outer peripheral groove 111A. That is, when the first operation wire 107A is wound from the neutral condition, the first outer peripheral groove 111A serves as a wire winding portion (first wire winding portion) around which the first operation wire 107A is wound. In this case, the first operation wire 107A is wound around the first inner peripheral groove 112A, and wound around the first outer peripheral groove 111A through the first relay groove 113A only one time. The first operation wire 107A then extends into the insertion section 2. Therefore, the first operation wire 107A is not wound around the first outer peripheral groove 111A twice.

On the other hand, as shown in FIG. 15B, if the pulley body 102 of the first pulley 101A is rotated clockwise when viewed from above in FIG. 12, the second body projection 116B of the pulley body 102 moves in the second inner peripheral groove 112B in the discharging direction of the second operation wire 107B, so that the second pulley component 104 does not, rotate together with the pulley body 102. In this case, in response to the rotation of the pulley body 102, the wire proximal end 37 of the second operation wire 107B moves in the second inner peripheral groove 112B in the discharging direction of the second operation wire 107B. As a result, a part of the second operation wire 107B wound around the second inner peripheral groove 112B in the neutral condition is discharged. In this case, the wire proximal end 37 of the second operation wire 107B is movable in the second inner peripheral groove 112B. Therefore, when the second operation wire 107B is slack, the wire proximal end 37 moves in the second inner peripheral groove 112B in the direction opposite to the discharging direction of the second operation wire 107B. As a result, the slack of the second operation wire 107B is absorbed. That is, the second inner peripheral groove 112B is provided with a slack absorbing portion 119B (second slack absorbing portion) configured to absorb the slack of the second operation wire 107B when the second operation wire 107B is discharged from the second inner peripheral groove 112B.

Thus, the bending device 100 having the above-described configuration provides the following advantageous effects. That is, the first pulley 101A and the second pulley 101B of the bending device 100 include the first inner peripheral groove 112A provided in the first pulley component 103 and the second inner peripheral groove 112B provided in the second pulley component 104 along the circumferential directions. The wire proximal end 37 of the first operation wire 107A is movable in the first inner peripheral groove 112A, and the wire proximal end 37 of the second operation wire 107B is movable in the second inner peripheral groove 112B. In the first pulley 101A and the second pulley 101B, if the pulley body 102 rotates from the neutral condition in one of the rotation directions, the first operation wire 107A is discharged. Contrarily, if the pulley body 102 rotates from the neutral condition in the other of the rotation directions, the second operation wire 107B is discharged. For example, when the first operation wire 107A is discharged, the first operation wire 107A may be slack. In this case, the wire proximal end 37 of the first operation wire 107A moves in the first inner peripheral groove 112A in the direction opposite to the discharging direction of the first operation wire 107A. As a result, the slack of the first operation wire 107A is absorbed. When the second operation wire 107B is discharged, the slack of the second operation wire 107B is absorbed in the same manner. As described above, in the bending device 100, the first pulley 101A and the second pulley 101B are provided with the spaces configured to absorb the slack of the operation wire 27. Thus, the slack of the operation wire 27 can be effectively absorbed without being affected by the design limitation of the operation section 3.

In the curving device 100, the first inner peripheral groove 112A and the second inner peripheral groove 112B are formed along the circumferential directions of the first pulley 101A and the second pulley 101B. This makes it possible to secure sufficient space (length) to absorb the slack of the operation wire 27. Thus, the slack of the long operation wire 27 can be sufficiently absorbed, which is advantageous in size reduction of the operation section 3.

Furthermore, in the bending device 100, one of the first pulley component 103 and the second pulley component 104 rotates together with the pulley body 102. When the first pulley component 103 rotates together with the pulley body 102, the first operation wire 107A is wound around the first outer peripheral groove 111A by the rotation of the first pulley component 103 together with the pulley body 102. In this case, the first operation wire 107A is wound around each of the first outer peripheral groove 111A and the first inner peripheral groove 112A one time. This can prevent, the first operation wire 107A from being wound around the first outer peripheral groove 111A twice. When the second pulley component 104 rotates together with the pulley body 102, it is also possible to prevent the second operation wire 107B from being wound around the second outer peripheral groove 111B twice.

### Modification of Third Embodiment

In the embodiment described above, both ends of the first inner peripheral groove 112A communicate with the first outer peripheral groove 111A, and both ends of the second inner peripheral groove 112B communicate with the second outer peripheral groove 111B. However, the present invention is not limited thereto. For example, only one end of the first inner peripheral groove 112A may communicate with the first outer peripheral groove 111A.

Furthermore, in the embodiment described above, the first operation wire 107A is wound in the counterclockwise direction when viewed from above in FIG. 12, and the second operation wire 107B is wound in the clockwise direction when viewed from above in FIG. 12. However, the second operation wire 107B has only to be configured to be wound in a direction opposite to a winding direction of the first operation wire 107A.

Still further, in the embodiment described above, in the neutral condition, the movement of the wire proximal end 37 of the first operation wire 107A in the direction opposite to the discharging direction of the first operation wire 107A is regulated by the collision of the press-bonded element 38 with the first component projection 118A. Similarly, in the neutral condition, the movement of the wire proximal end 37 of the second operation wire 107B in the direction opposite to the discharging direction of the second operation wire 107B is regulated by the collision of the press-bonded element 38 with the second component projection 118B. However, in the neutral condition, the wire proximal end 37 of the first operation wire 107A has only to be located in the first inner peripheral groove 112A so that its movement in the direction opposite to the discharging direction of the first operation wire 107A is regulated. Similarly, the wire proximal end 37 of the second operation wire 107B has only to be located in the second inner peripheral groove 112B so that its movement in the direction opposite to the discharging direction of the second operation wire 107B is regulated.

Still further, in the embodiment described above, the first component projection 118A of the first pulley component 103 is pressed by the first body projection 116A of the pulley body 102 so that the first pulley component 103 rotates together with the pulley body 102. Similarly, the second component projection 118B of the second pulley component 104 is pressed by the second body projection 116B of the pulley body 102 so that the second pulley component 104 rotates together with the pulley body 102. However, one of the first pulley component 103 and the second pulley component 104 has only to be configured to rotate together with the pulley body 102 if the pulley body 102 is rotated in one of the rotation directions from the neutral condition in which the bending section 7 is not bent, and the other of the first pulley component 103 and the second pulley component 104 has only to be configured to rotate together with the pulley body 102 if the pulley body 102 is rotated in the other of the rotation directions from the neutral condition.

### Fourth Embodiment

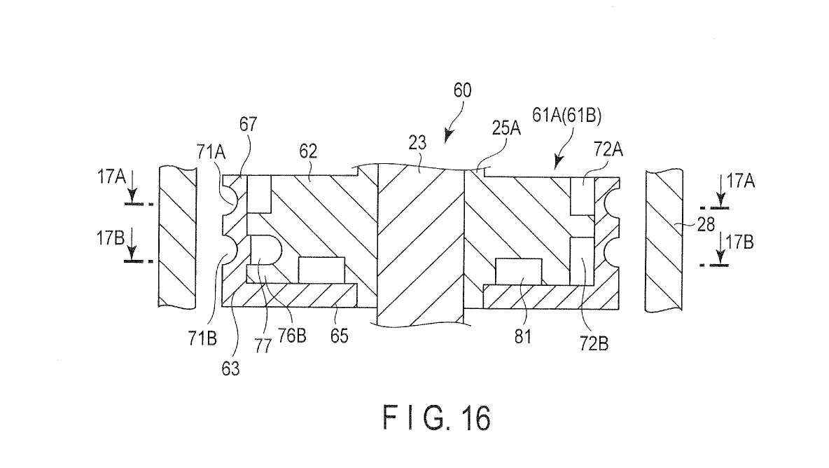

Now, a fourth embodiment of the present invention is described with reference to FIG. 16 to FIG. 20B. In the fourth embodiment, the configuration of the bending device 15 according to the first embodiment is modified as below. Components having the same parts and the same functions as those in the first embodiment are provided with the same reference signs and are not described.

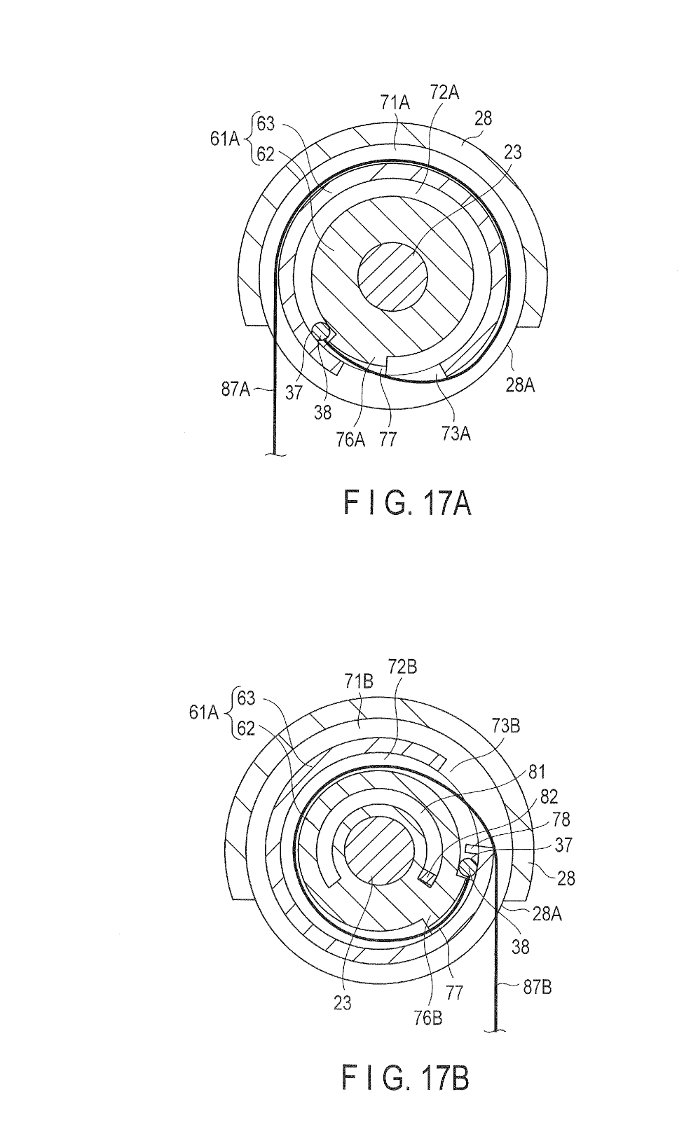

FIG. 16 to FIG. 17B are diagrams showing the configurations of a first pulley 61A and a first rotary cylindrical portion 25A of a bending device 60 according to the present embodiment. Although the first pulley 61A and the first rotary cylindrical portion 25A are described below, the same applies to a second pulley 61B and a second rotary cylindrical portion 25B.

As shown in FIG. 16 to FIG. 17B, the first pulley 61A includes a substantially columnar first pulley component (inner pulley component) 62 and a bottomed cylindrical second pulley component (outer pulley component) 63. The first pulley component 62 is formed integrally with the first rotary cylindrical portion 25A, and can rotate in directions around the axis of a first pulley 41A together with the first rotary cylindrical portion 25A. That is, the first pulley component 62 is a rotary portion configured to be rotated by the bending operation in a first bending operation knob 16A. The second pulley component 63 includes a bottom wall 65 provided to a lower side of the first pulley component 62, and a peripheral wall 67 provided to an outer peripheral side of the first pulley component 62. A first outer peripheral groove 71A and a second outer peripheral groove 71B are provided axially side by side in an outer peripheral surface of the peripheral wall 67 of the second pulley component 63. The first outer peripheral groove 71A and the second outer peripheral groove 71B are provided separately from each other in axial directions of the first pulley 61A.

Along circumferential directions of the first pulley 61A, a first inner peripheral groove 72A is provided at an upper end of the outer peripheral surface of the first component 62, and a second inner peripheral groove 72B is provided at a lower end of the outer peripheral surface of the first pulley component 62. The first inner peripheral groove 72A and the second inner peripheral groove 72B are provided separately from each other in the axial directions of the first pulley 61A. Outer peripheral walls of the first inner peripheral groove 72A and the second inner peripheral groove 72B are formed by the peripheral wall 67 of the second pulley component 63. That is, the first inner peripheral groove 72A and the second inner peripheral groove 72B are provided between the first pulley component 62 and the second pulley component 63. The first inner peripheral groove 72A is located to an inner peripheral side of the first outer peripheral groove 71A. The second inner peripheral groove 72B is located to the inner peripheral side of the second outer peripheral groove 71B. The peripheral wall 67 of the second pulley component 63 includes a first relay groove 73A which communicates the first outer peripheral groove 71A with the first inner peripheral groove 72A, and a second relay groove 73B which communicates the second outer peripheral groove 71B with the second inner peripheral groove 72B. In a neutral condition in which a bending section 7 is not bent, the first relay groove 73A and the second relay groove 73B are located out of phase with each other in the circumferential directions of the first pulley 61A. The first pulley component 62 includes a first pulley projection 76A provided in the first inner peripheral groove 72A, and a second pulley projection 76B provided in the second inner peripheral groove 72B. The first pulley projection 76A protrudes toward the outer peripheral side from an inner peripheral wall of the first inner peripheral groove 72A. Similarly, the second pulley projection 76B protrudes toward the outer peripheral side from an inner peripheral wall of the second inner peripheral groove 72B. The first pulley projection 76A and the second pulley projection 76B are provided with depressions 77. The second pulley component 63 is provided with a projection 78 protruding toward the inner peripheral side from the outer peripheral wall of the second inner peripheral groove 72B.

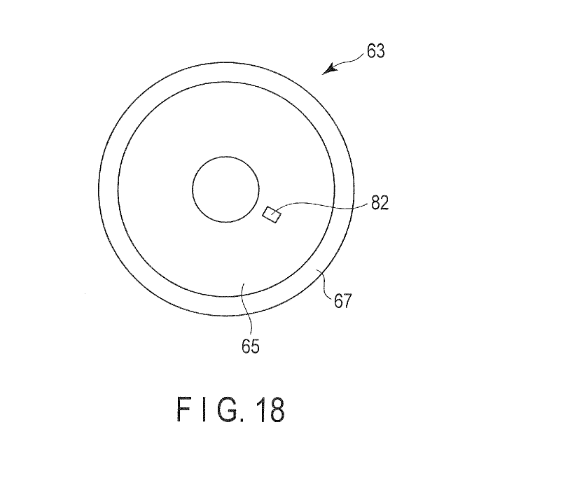

As shown in FIG. 16 and FIG. 17B, a link-rotating groove 81 is formed to the inner peripheral side of the second inner peripheral groove 72B on a lower surface of the first pulley component 62 along the directions around the axis of the first, pulley 61A. FIG. 18 is diagram showing the configuration of the second pulley component 63. As shown in FIG. 18, a link-rotating projection 82 is formed on an upper surface of the bottom wall 65 of the second pulley component 63. The link-rotating projection 82 engages with the link-rotating groove 81 (see FIG. 16). The link-rotating groove 81 is movable relative to the link-rotating projection 82 in the directions around the axis of the first pulley 61A. In the neutral condition in which the bending section 7 is not bent, the link-rotating projection 82 is located at a right end of the link-rotating groove 81 in FIG. 17B.

If the first rotary cylindrical portion 25A and the first pulley component 62 are rotated from the neutral condition in a counterclockwise direction (first rotation direction) when viewed from above in FIG. 16, the link-rotating projection 82 of the second pulley component 63 is pressed by the first pulley component 62. Thus, the second pulley component 63 rotates counterclockwise when viewed from above in FIG. 16 together with the first pulley component 62.

Contrarily, if the first rotary cylindrical portion 25A and the first pulley component 62 are rotated from the neutral condition in a clockwise direction (second rotation direction) when viewed from above in FIG. 16, the link-rotating groove 81 of the first pulley component 62 moves clockwise relative to the link-rotating projection 82. In this case, the link-rotating projection 82 of the second pulley component 63 does not rotate. Therefore, the first pulley component 62 alone rotates clockwise, and the second pulley component 63 does not rotate.

As shown in FIG. 17A, a wire proximal end 37 of a first operation wire 87A which is one of a pair of operation wires 27 connected to the first pulley 61A is located in the first inner peripheral groove 72A. A columnar press-bonded element 38 is fixed to the wire proximal end 37 of the first operation wire 87A. The wire proximal end 37 of the first operation wire 87A is movable in the first inner peripheral groove 72A.

In the neutral condition in which the bending section 7 is not bent, the wire proximal end 37 of the first operation wire 87A is located at an end of the first inner peripheral groove 72A to a left side of the first relay groove 73A in FIG. 17A. The first pulley projection 76A is located to a side where the first operation wire 87A is discharged of the wire proximal end 37. The first operation wire 87A is inserted through the depression 77 of the first pulley projection 76A. The first operation wire 87A inserted through the depression 77 passes through the first relay groove 73A and is then wound around the first cuter peripheral groove 71A only one time in a counterclockwise direction when viewed from above in FIG. 16. The first operation wire 87A then extends into an insertion section 2 from the first outer peripheral groove 71A. In this case, if the wire proximal end 37 moves to the first pulley projection 76A, the press-bonded element 38 collides with the first pulley projection 76A. As a result, the movement of the wire proximal end 37 beyond the first pulley projection 76A in the discharging direction of the first operation wire 87A is regulated. That is, the wire proximal end 37 of the first operation wire 87A in the neutral condition is located in the first inner peripheral groove 72A so that its movement in the discharging direction of the first operation wire 87A is regulated.

As shown in FIG. 17B, a wire proximal end 37 of a second operation wire 87B which is the other of the pair of operation wires 27 connected to the first pulley 61A is movable in the second inner peripheral groove 72B. A press-bonded element 38 is fixed to a wire proximal end 37 of the second operation wire 87B as in the first operation wire 87A.

In the neutral condition in which the bending section 7 is not bent, the wire proximal end 37 of the second operation wire 87B is located at an end of the second inner peripheral groove 72B to a lower side of the second relay groove 73B in FIG. 17B. The second pulley projection 76B is located to a side where the second operation wire 67B is discharged of the wire proximal end 37. The second operation wire 87B is inserted through the depression 77 of the second pulley projection 76B. The second operation wire 87B inserted through the depression 77 is wound around the second inner peripheral groove 72B only one time in a clockwise direction when viewed from above in FIG. 16. The second operation wire 87B then extends into the insertion section 2 from the second outer peripheral groove 71B through the second relay groove 73B. In this case, if the wire proximal end 37 moves to the projection 78, the press-bonded element 38 collides with the projection 78. As a result, the movement, of the wire proximal end 37 in a direction opposite to the discharging direction of the second operation wire 87A is regulated. That is, the wire proximal end 37 of the second operation wire 87B in the neutral condition is located in the second inner peripheral groove 72B so that its movement in the direction opposite to the discharging direction of the second operation wire 87B is regulated.

Now, the function of the bending device 60 according to the present embodiment is described. Although only the case where the bending section 7 is bent in the left-and-right directions by the first bending operation knob 16A is described below, the same applies to the case where the bending section 7 is bent in the up-and-down directions by a second bending operation knob 16B.

In order to bend the bending section 7 in the left-and-right directions, the operator rotates the first bending operation knob 16A, for example, in the direction of the arrow C in FIG. 2. Thus, the first rotary cylindrical portion 25A and the first pulley component 62 of the first pulley 61A rotate in the counterclockwise direction (first rotation direction) when viewed from above in FIG. 16.

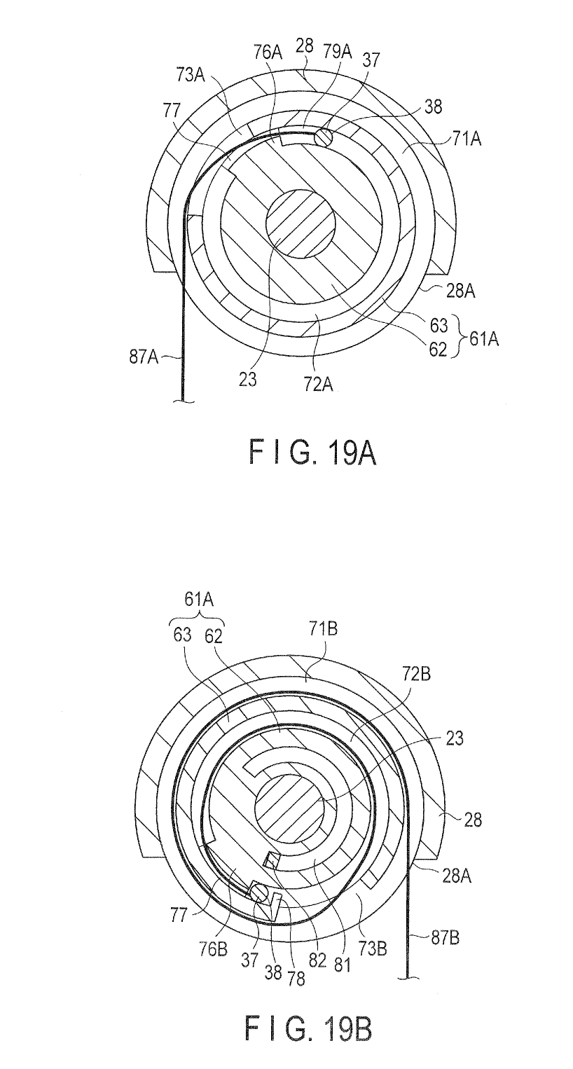

FIG. 19A and FIG. 19B show a conditions in which the first pulley component 62 of the first pulley 61A is rotated from the neutral condition in the counterclockwise direction when viewed from above in FIG. 16. As shown in FIG. 19B, when the first pulley component 62 of the first pulley 61A is rotated counterclockwise, the link-rotating projection 82 of the second pulley component 63 is pressed by the first pulley component 61. Thus, the second pulley component 63 rotates counterclockwise when viewed from above in FIG. 16 together with the first pulley component 62.

As shown in FIG. 19B, if the first pulley component 62 and the second pulley component 63 are rotated counterclockwise when viewed from above in FIG. 16, the second outer peripheral groove 71B rotates counterclockwise, and the second operation wire 87B is wound around the second outer peripheral groove 71B. That is, when the second operation wire 87B is wound from the neutral condition, the second outer peripheral groove 71B serves as a wire winding portion (second wire winding portion) around which the second operation wire 87B is wound. In this case, the second operation wire 87B is wound around the second inner peripheral groove 72B, and wound around the second outer peripheral groove 71B through the second relay groove 73B only one time. The second operation wire 87B then extends into the insertion section 2. Therefore, the second operation wire 87B is not wound around the second outer peripheral groove 71B twice.

On the other hand, as shown in FIG. 19A, when the first pulley component 62 and the second pulley component 63 are rotated counterclockwise, a part of the first operation wire 87A wound around the first outer peripheral groove 71A in the neutral condition is discharged. In this case, the wire proximal end 37 of the first operation wire 87A is movable in the first inner peripheral groove 72A. Therefore, when the first operation wire 37A is slack, the wire proximal end 37 moves in the first inner peripheral groove 72A in the direction opposite to the discharging direction of the first operation wire 87A. As a result, the slack of the first operation wire 87A is absorbed. That is, the first inner peripheral groove 72A is provided with a slack absorbing portion 79A (first slack absorbing portion) configured to absorb the slack of the first operation wire 87A when the first operation wire 87A is discharged from the first inner peripheral groove 72A.

In this way, the first operation wire 87A is discharged and the second operation wire 87B is wound from the neutral condition, so that the bending section. 7 bends in a predetermined direction (e.g. right direction).

In order to bend the bending section 7 in an opposite direction (e.g. left direction), the operator rotates the first bending operation knob 16A in the direction of the arrow C in FIG. 2. Thus, the first rotary cylindrical portion 25A and the first pulley component 62 of the first pulley 61A rotate in the clockwise direction (second rotation direction) when viewed from above in FIG. 16.

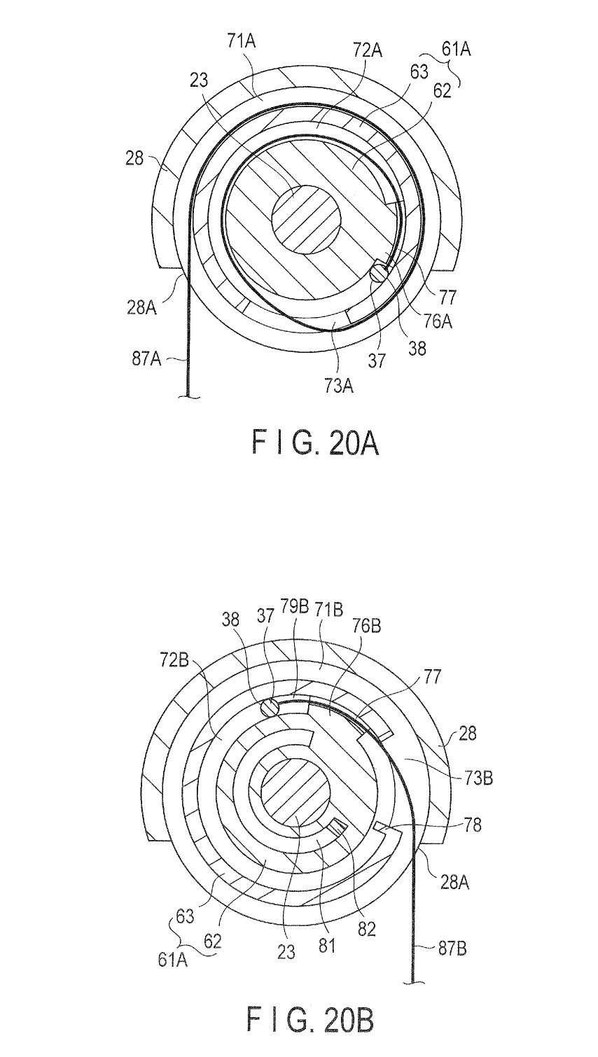

FIG. 20A and FIG. 20B show a conditions in which the first pulley component 62 of the first pulley 61A is rotated from the neutral condition in the clockwise direction when viewed from above in FIG. 16. As shown in FIG. 20B, when the first pulley component 62 of the first pulley 61A is rotated clockwise, the link-rotating groove 81 of the first pulley component 62 moves clockwise relative to the link-rotating projection 82. In this case, the link-rotating projection 82 of the second pulley component 63 does not rotate. Therefore, the first pulley component 62 alone rotates clockwise, and the second pulley component 63 does not rotate.

As shown in FIG. 20A, the first pulley component 62 alone rotates, so that the first pulley projection 76A of the first pulley component 62 moves in the first inner peripheral groove 72A in the direction opposite to the discharging direction of the first operation wire 87A. In this case, the movement of the wire proximal end 37 beyond the first pulley projection 76A in the discharging direction of the first operation wire 87A is regulated. Thus, in response to the movement of the first pulley projection 76A, the wire proximal end 37 of the first operation wire 87A moves in the first inner peripheral groove 72A in the direction opposite to the discharging direction of the first operation wire 87A together with the first pulley projection 76A. As a result, the first operation wire 87A is wound around the first inner peripheral groove 72A. That is, when the first operation wire 87A is wound from the neutral condition, the first inner peripheral groove 72A serves as a wire winding portion (first wire winding portion) around which the first operation wire 87A is wound. In this case, the first operation wire 87A is wound around the first inner peripheral groove 72A, and wound around the first outer peripheral groove 71A through the first relay groove 73A only one time. The first operation wire 87A then extends into the insertion section 2. Therefore, the first operation wire 87A is not wound around the first outer peripheral groove 71A twice.

On the other hand, as shown in FIG. 20B, if the first pulley component 62 alone is rotated clockwise when viewed from above in FIG. 16, a part of the second operation wire 87B wound around the second inner peripheral groove 72B in the neutral condition is discharged. In this case, the wire proximal end 37 of the second operation wire 87B is movable in the second inner peripheral groove 72B. Therefore, when the second operation wire 87B is slack, the wire proximal end 37 moves in the second inner peripheral groove 72B in the direction opposite to the discharging direction of the second operation wire 87B. As a result, the slack of the second operation wire 87B is absorbed. That is, the second inner peripheral groove 72B is provided with a slack absorbing portion 79B (second slack absorbing portion) configured to absorb the slack of the second operation wire 87B when the second operation wire 87B is discharged from the second inner peripheral groove 72B.

Thus, the bending device 60 having the above-described configuration provides the following advantageous effects. That is, the first pulley 61A and the second pulley 61B of the bending device 60 includes the first inner peripheral groove 72A provided in the upper surface of the first pulley component 62 and the second inner peripheral groove 72B provided in the lower surface of the first pulley component 62 along the circumferential directions. The wire proximal end 37 of the first operation wire 87A is movable in the first inner peripheral groove 72A, and the wire proximal end 37 of the second operation wire 87B is movable in the second inner peripheral groove 72B. In the first pulley 61A and the second pulley 61B, if the first pulley component 62 rotates from the neutral condition in one of the rotation directions, the second pulley component 63 rotates together with the first pulley component 62. Contrarily, if the first pulley component 62 rotates in the other of the rotation directions, the first pulley component 62 alone rotates, and the second pulley component 63 does not rotate. In the neutral condition, the first operation wire 87A is wound around the first outer peripheral groove 71A, and the second operation wire 87B is wound around the second inner peripheral groove 72B. If the first pulley component 62 and the second pulley component 63 rotate together in one of the rotation directions, the first operation wire 87A is discharged. If the first pulley component 62 alone rotates in the other of the rotation directions, the second operation wire 87B is discharged. When the first operation wire 87A is discharged, the first operation wire 87A may be slack. In this case, the wire proximal end 37 of the first operation wire 87A moves in the first inner peripheral groove 72A in the direction opposite to the discharging direction of the first operation wire 87A. As a result, the slack of the first operation wire 87A is absorbed. When the second operation wire 87B is discharged, the slack of the second operation wire 87B is absorbed in the same manner. As described above, in the bending device 60, the first pulley 61A and the second pulley 61B are provided with the spaces configured to absorb the slack of the operation wire 27. Thus, the slack of the operation wire 27 can be effectively absorbed without being affected by the design limitation of the operation section 3.

In the bending device 60, the first inner peripheral groove 72A and the second inner peripheral groove 72B are formed along the directions around the axes of the first pulley 61A and the second pulley 61B. This makes it possible to secure sufficient space to absorb the slack of the operation wire 27. Thus, the slack of the long operation wire 27 can be sufficiently absorbed, which is advantageous in size reduction of the operation section 3.

Furthermore, in the bending device 60, if the first, pulley component 62 and the second pulley component 63 rotate together in one of the rotation directions, the second operation wire 87B is wound around the second outer peripheral groove 71B. If the first pulley component 62 alone rotates in the other of the rotation directions, the first operation wire 87A is wound around the first inner peripheral groove 72A. In this case, the first operation wire 87A is wound around the first outer peripheral groove 71A and the first inner peripheral groove 72A. This can prevent the first operation wire 87A from being wound around the first outer peripheral groove 71A twice. It is likewise possible to prevent the second operation wire 87B from being wound around the second outer peripheral groove 71B twice.

Furthermore, in the first pulley 61A and the second pulley 61B, the first inner peripheral groove 72A and the second inner peripheral groove 72B are provided in the first pulley component 62. The second pulley component 63 is in a substantially bottomed cylindrical shape that covers the bottom surface and the outer peripheral surface of the first pulley component 62. Such a configuration allows the reduction of the axial dimensions of the first pulley 61A and the second pulley 61B.

### Modification of Fourth Embodiment

In the embodiment described above, both ends of the first inner peripheral groove 72A communicate with the first outer peripheral groove 71A, and both ends of the second inner peripheral groove 72B communicate with the second outer peripheral groove 71B. However, the present invention is not limited thereto. For example, at least one end of the first inner peripheral groove 72A may communicate with the first outer peripheral groove 71A.

Furthermore, in the embodiment described above, the first operation wire 87A is wound in the counterclockwise direction when viewed from above in FIG. 16, and the second operation wire 87B is wound in the clockwise direction when viewed from above in FIG. 16. However, the second operation wire 87B has only to be configured to be wound in a direction opposite to a winding direction of the first operation wire 87A.

Still further, in the embodiment described above, in the neutral condition, the movement of the wire proximal end 37 of the first operation wire 87A in the discharging direction of the first operation wire 87A is regulated by the collision of the press-bonded element 38 with the first pulley projection 76A. However, in the neutral condition, the movement of the wire proximal end 37 of the first operation wire 87A in the discharging direction of the first operation wire 87A has only to be configured to be regulated. Similarly, in the neutral condition, the movement of the wire proximal end 37 of the second operation wire 87B in the direction opposite to the discharging direction of the second operation wire 87B has only to be configured to be regulated.

Still further, in the embodiment described above, the second pulley component 63 includes the bottom wall 65 provided to the lower side of the first pulley component 62, and the peripheral wall 67 provided to the outer peripheral side of the first pulley component 62. However, instead of the bottom wall 65, an upper wall may be provided to an upper side of the first pulley component 62. In this case, the link-rotating groove 81 is provided on an upper surface of the first pulley component 62, and the link-rotating projection 82 is provided on the upper wall of the second pulley component 63. Alternatively, the second pulley component 63 may be in a substantially cylindrical shape that only consists of the peripheral wall 67. In this case, the link-rotating groove 81 is provided in the outer peripheral surface of the first pulley component 62, and the link-rotating projection 82 is provided on an inner peripheral surface of the peripheral wall 67 of the second pulley component 63.

Still further; in the embodiment described above, when the link-rotating projection 82 of the second pulley component 63 is pressed by the first pulley component 62, the second pulley component 63 rotates together with the first pulley component 62. When the link-rotating groove 81 of the first pulley component 62 moves relative to the link-rotating projection 82, the first pulley component 62 alone rotates. However, the second pulley component 63 has only to be configured to rotate together with the first pulley component 62 if the first pulley component 62 is rotated in one of the rotation directions from the neutral condition, and the first pulley component 62 alone has only to be configured to rotate if the first pulley component 62 is rotated in the other of the rotation directions from the neutral condition.

(Other Modifications)

In the embodiments described above, the bending operation transmission mechanism 20 includes two pulleys, and the bending section 7 is bent in the left-and-right directions and up-and-down directions. However, the bending operation transmission mechanism 20 may be provided with only one pulley. In this case, the bending section 7 is bent in the left-and-right directions or up-and-down directions.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An endoscope bending device comprising:
an endoscope insertion section which includes a bending section configured to bend;
a bending operation section which is provided to a proximal side of the endoscope insertion section, and which is configured to perform a bending operation of the bending section;
a pulley which includes a rotary portion configured to be rotated in a first rotation direction and in a second rotation direction opposite to the first rotation direction by the bending operation in the bending operation section, an outer peripheral groove provided in an outer peripheral surface along circumferential directions, an inner peripheral groove provided along the circumferential directions to an inner peripheral side of the outer peripheral groove, and a relay groove communicating the outer peripheral groove with the inner peripheral groove;
an operation wire which includes a wire proximal end movably provided in the inner peripheral groove of the pulley, and a wire distal end connected to the bending section, the operation wire extending into the endoscope insertion section after being wound around the outer peripheral groove or the inner peripheral groove in a neutral condition in which the bending section is not bent, the rotary portion of the pulley rotating from the neutral condition so that the operation wire is wound around the pulley or discharged from the pulley, and thereby the operation wire bending the bending section;
a wire winding portion which is configured to further wind the operation wire around the outer peripheral groove or the inner peripheral groove when the operation wire is wound from the neutral condition; and
a slack absorbing portion which is configured to absorb the slack of the operation wire by moving the wire proximal end of the operation wire in the inner peripheral groove in a direction opposite to a discharging direction of the operation wire during the operation of discharging the operation wire from the neutral condition.

2. The endoscope bending device according to claim 1, wherein
the outer peripheral groove includes a first outer peripheral groove, and a second outer peripheral groove which is provided separately from the first outer peripheral groove in axial directions of the pulley,
the inner peripheral groove includes a first inner peripheral groove, and a second inner peripheral groove which is provided separately from the first inner peripheral groove in the axial directions of the pulley,
the relay groove includes a first relay groove which communicates the first outer peripheral groove with the first inner peripheral groove, and a second relay groove which communicates the second outer peripheral groove with the second inner peripheral groove,
the operation wire includes
a first operation wire the wire proximal end of which is provided in the first inner peripheral groove, and which is wound around the first outer peripheral groove through the first relay groove in the neutral condition and then extends into the endoscope insertion section from the first outer peripheral groove, the first operation wire being configured to be discharged by the rotation of the rotary portion of the pulley in the first rotation direction from the neutral condition, and the first operation wire being configured to be wound by the rotation of the rotary portion of the pulley in the second rotation direction from the neutral condition, and
a second operation wire the wire proximal end of which is provided in the second inner peripheral groove, and which is wound around the second outer peripheral groove through the second relay groove in the neutral condition in a direction opposite to a winding direction of the first operation wire and then extends into the endoscope insertion section from the second outer peripheral groove, the second operation wire being configured to be wound by the rotation of the rotary portion of the pulley in the first rotation direction from the neutral condition, and the second operation wire being configured to be discharged by the rotation of the rotary portion of the pulley in the second rotation direction from the neutral condition,
the wire winding portion includes a first wire winding portion which is configured to further wind the first operation wire around the first outer peripheral groove when the first operation wire is wound from the neutral condition, and a second wire winding portion which is configured to further wind the second operation wire around the second outer peripheral groove when the second operation wire is wound from the neutral condition, and
the slack absorbing portion includes
a first slack absorbing portion which is configured to absorb the slack of the first operation wire by moving the wire proximal end of the first operation wire in the first inner peripheral groove in the direction opposite to the discharging direction of the first operation wire during the operation of discharging the first operation wire from the neutral condition, and
a second slack absorbing portion which is configured to absorb the slack of the second operation wire by moving the wire proximal end of the second operation wire in the second inner peripheral groove in the direction opposite to the discharging direction of the second operation wire during the operation of discharging the second operation wire from the neutral condition.

3. The endoscope bending device according to claim 1, wherein
the pulley includes
a first pulley component which is configured to rotate together with the rotary portion during the rotation of the rotary portion in the first rotation direction from the neutral condition, and which is configured to not rotate during the rotation of the rotary portion in the second rotation direction from the neutral condition, and
a second pulley component which is configured to not rotate during the rotation of the rotary portion in the first rotation direction from the neutral condition, and which is configured to rotate together with the rotary portion during the rotation of the rotary portion in the second rotation direction from the neutral condition,
the outer peripheral groove includes a first outer peripheral groove provided in an outer peripheral surface of the first pulley component, and a second outer peripheral groove provided in an outer peripheral surface of the second pulley component,
the inner peripheral groove includes first, inner peripheral groove provided between the rotary portion and the first pulley component, and a second inner peripheral groove provided between the rotary portion and the second pulley component,
the relay groove includes a first relay groove which communicates the first outer peripheral groove with the first inner peripheral groove, and a second relay groove which communicates the second outer peripheral groove with the second inner peripheral groove,
the operation wire includes
a first operation wire which is configured to be discharged by the rotation of the rotary portion of the pulley in the first rotation direction from the neutral condition, and which is configured to be wound by the rotation of the rotary portion of the pulley in the second rotation direction from the neutral condition, the wire proximal end of the first operation wire being provided in the first inner peripheral groove so that the movement thereof in the discharging direction of the first operation wire is regulated in the neutral condition, and the first operation wire being wound around the first outer peripheral groove through the first relay groove and then extending into the endoscope insertion section from the first outer peripheral groove, and
a second operation wire which is configured to be wound by the rotation of the rotary portion of the pulley in the first rotation direction from the flout ref condition, and which is configured to be discharged by the rotation of the rotary portion of the pulley in the second rotation direction from the neutral condition, the wire proximal end of the second operation wire being provided in the second inner peripheral groove so that the movement thereof in the discharging direction of the second operation wire is regulated in the neutral condition, and the second operation wire being wound around the second outer peripheral groove through the second relay groove in a direction opposite to a winding direction of the first operation wire and then extending into the endoscope insertion section from the second outer peripheral groove, and
the wire winding portion includes
a first wire winding portion provided in the rotary portion, the first wire winding portion being configured to move the wire proximal end of the first operation wire in the direction opposite to the discharging direction of the first operation wire and to wind the first operation wire around the first inner peripheral groove during the operation of winding the first operation wire from the neutral condition, and
a second wire winding portion provided in the rotary portion, the second wire winding portion being configured to move the wire proximal end of the second operation wire in the direction opposite to the discharging direction of the second operation wire and to wind the second operation wire around the second inner peripheral groove during the operation of winding the second operation wire from the neutral condition.

4. The endoscope bending device according to claim 3, wherein
the slack absorbing portion includes
a first slack absorbing portion which is configured to absorb the slack of the first operation wire by moving the wire proximal end of the first operation wire in the first inner peripheral groove in the direction opposite to the discharging direction of the first operation wire during the operation of discharging the first operation wire from the neutral condition, and
a second slack absorbing portion which is configured to be absorb the slack of the second operation wire by moving the wire proximal end of the second operation wire in the second inner peripheral groove in the direction opposite to the discharging direction of the second operation wire during the operation of discharging the second operation wire from the neutral condition.

5. The endoscope bending device according to claim 1, wherein
the pulley includes
a first pulley component which is configured to not rotate during the rotation of the rotary portion in the first rotation direction from the neutral condition, and which is configured to rotate together with the rotary portion during the rotation of the rotary portion in the second rotation direction from the neutral condition, and
a second pulley component which is configured to rotate together with the rotary portion during the rotation of the rotary portion in the first rotation direction from the neutral condition, and which is configured to not rotate during the rotation of the rotary portion in the second rotation direction from the neutral condition,
the outer peripheral groove includes a first outer peripheral groove provided in an outer peripheral surface of the first pulley component, and a second outer peripheral groove provided in an outer peripheral surface of the second pulley component,
the inner peripheral groove includes a first inner peripheral groove provided between the rotary portion and the first pulley component, and a second inner peripheral groove provided between the rotary portion and the second pulley component,
the relay groove includes a first relay groove which communicates the first outer peripheral groove with the first inner peripheral groove, and a second relay groove which communicates the second outer peripheral groove with the second inner peripheral groove,
the operation wire includes
a first operation wire which is configured to be discharged by the rotation of the rotary portion of the pulley in the first rotation direction from the neutral condition, and which is configured to be wound by the rotation of the rotary portion of the pulley in the second rotation direction from the neutral condition, the wire proximal end of the first operation wire being provided in the first inner peripheral groove so that the movement thereof in the direction opposite to the discharging direction of the first operation wire is regulated in the neutral condition, and the first operation wire being wound around the first inner peripheral groove and then extending into the endoscope insertion section from the first outer peripheral groove through the first relay groove, and
a second operation wire which is configured to be wound by the rotation of the rotary portion of the pulley in the first rotation direction from the neutral condition, and which is configured to be discharged by the rotation of the rotary portion of the pulley in the second rotation direction from the neutral condition, the wire proximal end of the second operation wire being provided in the second inner peripheral groove so that the movement thereof in the direction opposite to the discharging direction of the second operation wire is regulated in the neutral condition, and the second operation wire being wound around the second inner peripheral groove in a direction opposite to a winding direction of the first operation wire and then extending into the endoscope insertion section from the second outer peripheral groove through the second relay groove, and
the wire winding portion includes
a first wire winding portion provided in the first pulley component, the first wire winding portion being configured to wind the first operation wire around the first outer peripheral groove during the operation of winding the first operation wire from the neutral condition, and
a second wire winding portion provided in the second pulley component, the second wire winding portion being configured to wind the second operation wire around the second outer peripheral groove during the operation of winding the second operation wire from the neutral condition.

6. The endoscope bending device according to claim 5, wherein
the slack absorbing portion includes
a first slack absorbing portion which is configured to absorb the slack of the first operation wire by moving the wire proximal end of the first operation wire in the first inner peripheral groove in the direction opposite to the discharging direction of the first operation wire during the operation of discharging the first operation wire from the neutral condition, and
a second slack absorbing portion which is configured to absorb the slack of the second operation wire by moving the wire proximal end of the second operation wire in the second inner peripheral groove in the direction opposite to the discharging direction of the second operation wire during the operation of discharging the second operation wire from the neutral condition.

7. The endoscope bending device according to claim 1, wherein
the pulley includes an outer pulley component which includes a peripheral wall provided on an outer peripheral side of the rotary portion, the outer pulley component being configured to rotate together with the rotary portion during the rotation of the rotary portion in the first rotation direction from the neutral condition, and the outer pulley component being configured to not rotate during the rotation of the rotary portion in the second rotation direction from the neutral condition,
the outer peripheral groove includes a first outer peripheral groove provided in an outer peripheral surface of the cuter pulley component, and a second outer peripheral groove provided in the outer peripheral surface of the outer pulley component separately from the first outer peripheral groove in axial directions of the pulley,
the inner peripheral groove includes a first inner peripheral groove provided between the rotary portion and the outer pulley component, and a second inner peripheral groove provided between the rotary portion and the outer pulley component separately from the first inner peripheral groove in the axial directions of the pulley,
the relay groove includes a first relay groove which communicates the first outer peripheral groove with the first inner peripheral groove, and a second relay groove which communicates the second outer peripheral groove with the second inner peripheral groove,
the operation wire includes
a first operation wire which is configured to be discharged by the rotation of the rotary portion of the pulley in the first rotation direction from the neutral condition, and which is configured to be wound by the rotation of the rotary portion of the pulley in the second rotation direction from the neutral condition, the wire proximal end of the first operation wire being provided in the first inner peripheral groove so that the movement thereof in the discharging direction of the first operation wire is regulated in the neutral condition, and the first operation wire being wound around the first outer peripheral groove through the first relay groove and then extending into the endoscope insertion section from the first outer peripheral groove, and
a second operation wire which is configured to be wound by the rotation of the rotary portion of the pulley in the first rotation direction from the neutral condition, and which is configured to be discharged by the rotation of the rotary portion of the pulley in the second rotation direction from the neutral condition, the wire proximal end of the second operation wire being provided in the second inner peripheral groove so that the movement thereof in the direction opposite to the discharging direction of the second operation wire is regulated in the neutral condition, and the second operation, wire being wound around the second inner peripheral groove in a direction opposite to a winding direction of the first operation wire and then extending into the endoscope insertion section from the second outer peripheral groove through the second relay groove, and
the wire winding portion includes
first wire winding portion provided in the rotary portion, the first wire winding portion being configured to move the wire proximal end of the first operation wire in the direction opposite to the discharging direction of the first operation wire and to wind the first operation wire around the first inner peripheral groove during the operation of winding the first operation wire from the neutral condition, and
a second wire winding portion provided in the outer pulley component, the second wire winding portion being configured to wind the second operation wire around the second outer peripheral groove during the operation of winding the second operation wire from the neutral condition.

8. The endoscope bending device according to claim 7, wherein
the slack absorbing portion includes
a first slack absorbing portion which is configured to absorb the slack of the first operation wire by moving the wire proximal end of the first operation wire in the first inner peripheral groove in the direction opposite to the discharging direction of the first operation wire during the operation of discharging the first operation wire from the neutral condition, and
a second slack absorbing portion which is configured to absorb the slack of the second operation wire by moving the wire proximal end of the second operation wire in the second inner peripheral groove in the direction opposite to the discharging direction of the second operation wire during the operation of discharging the second operation wire from the neutral condition.
